(19) United States
(12) Patent Application Publication
(10) Pub. No.: US 2025/0179571 A1
(43) Pub. Date: Jun. 5, 2025

(54) METHODS AND COMPOSITIONS FOR ANALYTE DETECTION USING DILUTED READOUT SIGNALS

(71) Applicant: 10x Genomics, Inc., Pleasanton, CA (US)

(72) Inventor: Hiroshi SASAKI, Dublin, CA (US)

(73) Assignee: 10x Genomics, Inc., Pleasanton, CA (US)

(21) Appl. No.: 18/132,308

(22) Filed: Apr. 7, 2023

Related U.S. Application Data
(60) Provisional application No. 63/329,335, filed on Apr. 8, 2022.

(51) Int. Cl. C12Q 1/6876; C12Q 1/6806; C12Q 1/682; C12Q 1/6825; C12Q 1/6844

(52) U.S. Cl. CPC C12Q 1/6876; C12Q 1/6806; C12Q 1/682; C12Q 1/6825; C12Q 1/6844; C12Q 2600/16

(57) ABSTRACT

In some aspects, the present disclosure generally relates to methods and compositions for detecting a plurality of molecules of one or more analytes in a sample. In some aspects, the present disclosure relates to methods for determining the locations and identities of analytes in a biological sample using detectable probes. In some aspects, the present disclosure relates to methods for reducing the crowding of signals that are used for determining the locations of the analytes (e.g., spot-calling signals). The methods herein have particular applicability in the detection of spot-calling signals and barcode sequences in sequencing-by-hybridization (SBH) methods, including those relying on combinatorial labeling schemes and decoding of the barcodes by sequential cycles of spot-calling and/or decoding using hybridization probes.

### CROSS-REFERENCE TO RELATED APPLICATIONS

This application claims priority to U.S. Provisional Patent Application No. U.S. 63/329,335, filed Apr. 8, 2022, entitled “METHODS AND COMPOSITIONS FOR ANALYTE DETECTION USING DILUTED READOUT SIGNALS,” which is herein incorporated by reference in its entirety for all purposes.

### FIELD

The present disclosure generally relates to methods and compositions for detecting a plurality of molecules of one or more analytes in a sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays are important tools, for example, for understanding the molecular basis of cell identity and developing treatment for diseases. Certain existing in situ analyte detection methods can yield detection signals outside of an optimal range. For example, signals with large size and/or high intensity may result in optical overcrowding, reducing the quality of image analysis. There is a need for new and improved methods for in situ assays. The present disclosure addresses these and other needs.

### SUMMARY

In some embodiments, provided herein is a method for analyzing a biological sample, comprising: (a) contacting the biological sample with a plurality of primary probes, wherein: each primary probe comprises (i) a binding region configured to bind to an analyte, and (ii) a barcode region associated with the analyte; (b) contacting the biological sample with a library of intermediate probes, wherein: each intermediate probe comprises (i) a hybridization region configured to hybridize to the barcode region of a primary probe of the plurality of primary probes or a complement of the barcode region, (ii) a spot-calling region, and optionally (iii) a decoding region; and two or more of the intermediate probes in the library comprise a common spot-calling region; (c) detecting signals associated with the spot-calling regions of the library of intermediate probes at multiple locations in the biological sample, thereby detecting the locations of the analytes associated with the barcode regions or complements thereof hybridized by the library of intermediate probes; and (d) detecting signals or absence thereof associated with the barcode regions or complements thereof at at least a subset of the locations detected in step (c).

In any of the embodiments herein, in (b), the hybridization region of the intermediate probe can hybridize to the barcode region of the primary probe. In any of the embodiments herein, the method can comprise one or more wash steps after (a), optionally wherein the one or more wash steps comprise stringent wash to remove unbound and/or nonspecifically bound primary probe molecules from the analytes in the biological sample. In any of the embodiments herein, the method can comprise one or more wash steps after (b), optionally wherein the one or more wash steps comprise stringent wash to remove unbound and/or nonspecifically bound intermediate probe molecules from the primary probes in the biological sample.

In any of the embodiments herein, in (b), the hybridization region of the intermediate probe can hybridize to the complement of the barcode region. In any of the embodiments herein, the method can comprise one or more wash steps after (a), optionally wherein the one or more wash steps comprise stringent wash to remove unbound and/or nonspecifically bound primary probe molecules from the analytes in the biological sample.

In any of the embodiments herein, the method can comprise a step of generating a product of the primary probe after (a), wherein the product comprises the complement of the barcode region of the primary probe. In any of the embodiments herein, the product can be a rolling circle amplification (RCA) product comprising multiple copies of the complement of the barcode region. In any of the embodiments herein, the method can comprise one or more wash steps after (b), optionally wherein the one or more wash steps comprise stringent wash to remove unbound and/or nonspecifically bound intermediate probe molecules from the products of the primary probes in the biological sample.

In any of the embodiments herein, the detecting in (c) can comprise detecting signals associated with detectable probes that are hybridized to the spot-calling regions in the library of intermediate probes, optionally wherein the detectable probes are fluorescently labeled and/or configured to directly or indirectly bind to fluorescently labeled probes. In any of the embodiments herein, the detecting in (d) can comprise: detecting signals associated with detectable probes that are hybridized to the barcode regions or complements thereof; and/or detecting signals associated with detectable probes that are hybridized to the intermediate probes which are in turn hybridized to the barcode regions or complements thereof, optionally wherein the detectable probes are fluorescently labeled and/or configured to directly or indirectly bind to fluorescently labeled probes. In any of the embodiments herein, the detecting in (d) can comprise detecting signals associated with the decoding regions of the library of intermediate probes.

In any of the embodiments herein, all of the intermediate probes in the library can comprise the same common spot-calling region.

In any of the embodiments herein, the library can comprise a first sub-library and a second sub-library, the intermediate probes in the first sub-library can comprise a first common spot-calling region, the intermediate probes in the second sub-library can comprise a second common spot-calling region, and the first and second common spot-calling regions can be different.

In any of the embodiments herein, the detecting in (c) can comprise: (i) detecting signals associated with the first common spot-calling region of the first sub-library of intermediate probes at multiple locations in the biological sample, thereby detecting the locations of the analytes associated with the barcode regions or complements thereof hybridized by the first sub-library of intermediate probes; and/or (ii) detecting signals associated with the second common spot-calling region of the second sub-library of intermediate probes at multiple locations in the biological sample, thereby detecting the locations of the analytes associated with the barcode regions or complements thereof hybridized by the second sub-library of intermediate probes. In any of the embodiments herein, the detecting in (c) can comprise: contacting the biological sample with detectable probes that hybridize to the first common spot-calling regions in the first sub-library of intermediate probes; and/or contacting the biological sample with detectable probes that hybridize to the second common spot-calling regions in the second sub-library of intermediate probes. In any of the embodiments herein, the detecting in (d) can comprise: detecting signals associated with the barcode regions or complements thereof at at least a subset of the locations detected in (c)(i); and/or detecting signals associated with the barcode regions or complements thereof at at least a subset of the locations detected in (c)(ii).

In any of the embodiments herein, the first sub-library can comprise intermediate probes configured to hybridize to primary probes or complements thereof associated with two or more different analytes; and/or the second sub-library can comprise intermediate probes configured to hybridize to primary probes or complements thereof associated with two or more different analytes.

In any of the embodiments herein, one or more intermediate probes in the first sub-library and one or more intermediate probes in the second sub-library can be configured to hybridize to primary probes or complements thereof associated with different analytes.

In any of the embodiments herein, the method can comprise contacting the biological sample with a library of decoding intermediate probes, wherein each decoding intermediate probe comprises (i) a hybridization region configured to hybridize to the barcode region of a primary probe of the plurality of primary probes or a complement of the barcode region, and (ii) a decoding region.

In any of the embodiments herein, the method can comprise contacting the biological sample with detectable probes that hybridize to the decoding regions of the library of decoding intermediate probes.

In any of the embodiments herein, the method can comprise detecting signals or absence thereof associated with the detectable probes, optionally wherein the detectable probes are fluorescently labeled and/or configured to directly or indirectly bind to fluorescently labeled probes.

In any of the embodiments herein, the method can comprise contacting the biological sample with a subsequent library of decoding intermediate probes, wherein each subsequent decoding intermediate probe comprises (i) a hybridization region configured to hybridize to the barcode region of a primary probe of the plurality of primary probes or a complement of the barcode region, and (ii) a subsequent decoding region.

In any of the embodiments herein, the method can comprise contacting the biological sample with subsequent detectable probes that hybridize to the subsequent decoding regions of the subsequent library of decoding intermediate probes, and detecting signals or absence thereof associated with the subsequent detectable probes.

In any of the embodiments herein, the decoding intermediate probes and the subsequent decoding intermediate probes can hybridize to the same barcode sequence (or complement thereof) or different barcode sequences (or complements thereof) in the barcode region, optionally wherein the different barcode sequences are partially overlapping.

In any of the embodiments herein, the decoding region and the subsequent decoding region can be configured to hybridize to the same detectable probe, optionally wherein the decoding region and the subsequent decoding region are identical in sequence and are hybridized to detectable probes comprising the same detectable label. In any of the embodiments herein, the decoding region and the subsequent decoding region can be configured to hybridize to detectable probes of different sequences, optionally wherein each detectable probes of a different sequence comprises a different detectable label.

In any of the embodiments herein, the intermediate probes in the library of intermediate probes in (b) can comprise the decoding region.

In any of the embodiments herein, the method can comprise contacting the biological sample with detectable probes that hybridize to the decoding regions of the library of intermediate probes.

In any of the embodiments herein, the method can comprise detecting signals or absence thereof associated with the detectable probes, optionally wherein the detectable probes are fluorescently labeled and/or configured to directly or indirectly bind to fluorescently labeled probes.

In any of the embodiments herein, the method can comprise generating a signal code sequence for one or more of the multiple locations, the signal code sequence comprising signal codes corresponding to: the signals (or absence thereof) associated with the detectable probes hybridized to the decoding regions of the library of intermediate probes, the signals (or absence thereof) associated with the detectable probes hybridized to the decoding regions of the library of decoding intermediate probes, and/or the signals (or absence thereof) associated with the subsequent detectable probes hybridized to the decoding regions of the library of subsequent decoding intermediate probes, wherein the signal code sequence corresponds to an analyte, thereby detecting the analyte at the one or more of the multiple locations.

In any of the embodiments herein, the barcode region can comprise one or more barcode sequence or a combination of barcode sequences, and wherein the barcode sequence or the combination of barcode sequences corresponds to the analyte associated with the barcode region.

In any of the embodiments herein, each primary probe can be independently selected from the group consisting of: a primary probe comprising a 3′ or 5′ overhang, optionally wherein the 3′ or 5′ overhang comprises one or more barcode sequences; a primary probe comprising a 3′ overhang and a 5′ overhang, optionally wherein the 3′ overhang and the 5′ overhang each independently comprises one or more barcode sequences; a circular primary probe; and a circularizable primary probe or probe set.

In any of the embodiments herein, each intermediate probe can be independently selected from the group consisting of: an intermediate primary probe comprising a 3′ or 5′ overhang, optionally wherein the 3′ or 5′ overhang comprises one or more spot-calling regions and/or decoding regions; an intermediate probe comprising a 3′ overhang and a 5′ overhang, optionally wherein the 3′ overhang and the 5′ overhang each independently comprise one or more spot-calling regions and/or decoding regions; a circular intermediate probe; and a circularizable intermediate probe or probe set.

In any of the embodiments herein, the analyte can comprise a nucleic acid, a protein, a carbohydrate, a lipid, or a small molecule, or a complex thereof. In any of the embodiments herein, the analyte can be a genomic DNA, cDNA, or mRNA, and the primary probe can hybridize to the genomic DNA, cDNA, or mRNA. In any of the embodiments herein, the analyte can be a protein, and the primary probe can comprise a binding region and one or more reporter oligonucleotides conjugated thereto, wherein the binding region binds the protein and the one or more reporter oligonucleotides comprise the barcode region.

In any of the embodiments herein, the biological sample can be non-homogenized and optionally selected from the group consisting of a formalin-fixed, paraffin-embedded (FFPE) sample, a frozen tissue sample, and a fresh tissue sample. In any of the embodiments herein, the biological sample can be fixed or not fixed. In any of the embodiments herein, the biological sample can be permeabilized. In any of the embodiments herein, the biological sample can be embedded in a matrix, optionally wherein the matrix comprises a hydrogel. In any of the embodiments herein, the biological sample can be cleared, optionally wherein the clearing comprises contacting the biological sample with a proteinase. In any of the embodiments herein, the biological sample can be crosslinked. In any of the embodiments herein, the biological sample can be a tissue slice between about 1 μm and about 50 μm in thickness, optionally wherein the tissue slice is between about 5 μm and about 35 μm in thickness.

In some embodiments, provided herein is a method for analyzing a biological sample, comprising: (a) contacting the biological sample with a plurality of primary probes, wherein: each primary probe comprises (i) a binding region configured to bind to an analyte, and (ii) a barcode region associated with the analyte; and each primary probe is circular or circularizable; (b) generating rolling circle amplification (RCA) products of the primary probes, wherein each RCA product comprises a complement of the barcode region of the primary probe; (c) contacting the biological sample with a library of intermediate probes, wherein: each intermediate probe comprises (i) a hybridization region configured to hybridize to the complement of the barcode region of a primary probe, (ii) a spot-calling region, and (iii) a decoding region; and two or more of the intermediate probes in the library comprise a common spot-calling region; (d) detecting signals associated with the spot-calling regions of the library of intermediate probes at multiple locations in the biological sample, thereby detecting the locations of the analytes associated with the complements of the barcode regions hybridized by the library of intermediate probes; and (e) detecting signals or absence thereof associated with the decoding regions of the library of intermediate probes at at least a subset of the locations detected in step (d).

In any of the embodiments herein, all of the intermediate probes in the library can comprise the same common spot-calling region.

In any of the embodiments herein, the library can comprise a first sub-library and a second sub-library; the intermediate probes in the first sub-library can comprise a first common spot-calling region; the intermediate probes in the second sub-library can comprise a second common spot-calling region; and the first and second common spot-calling regions can be different.

In any of the embodiments herein, a unique signal code sequence can be assigned to each analyte to be analyzed; each signal code sequence can be derivable by interrogating the complements of the barcode regions with sequential intermediate probe libraries; the method can further comprise one or more sequential cycles, each cycle comprising: (i) contacting the biological sample with a subsequent intermediate probe library, wherein each intermediate probe in the subsequent intermediate probe library comprises (a) a hybridization region configured to hybridize to the complement of the barcode region of a primary probe, (b) a decoding region, and optionally (c) a spot-calling region; (ii) optionally detecting signals associated with the spot-calling region of the subsequent intermediate probe library at multiple locations in the biological sample, thereby detecting the locations of the analytes associated with the complements of the barcode regions hybridized by the subsequent intermediate probe library; and (iii) detecting signals or absence thereof associated with the decoding regions of intermediate probes in the subsequent intermediate probe library at at least a subset of the locations detected in step (c) and/or step (ii); a sequence of signals sequentially detected at at least a subset of the multiple locations in the biological sample in the sequential cycles can delineate a signal code sequence corresponding to a particular analyte, thereby revealing the location of the analyte in the biological sample; and the locations of one or more of the analytes in the biological sample can be determined.

In some embodiments, provided herein is a method for analyzing a biological sample, comprising: (a) contacting the biological sample with a plurality of primary probes, wherein: each primary probe comprises (i) a binding region configured to bind to an analyte, and (ii) a barcode region associated with the analyte; each primary probe is circular or circularizable; (b) generating rolling circle amplification (RCA) products of the primary probes, wherein each RCA product comprises a complement of the barcode region of the primary probe; (c) contacting the biological sample with a first library of intermediate probes for a first plurality of analytes, wherein: each intermediate probe of the first library comprises (i) a hybridization region configured to hybridize to the complement of the barcode region of a primary probe, (ii) a first common spot-calling region, and (iii) a decoding region; (d) detecting signals associated with the first common spot-calling regions at first multiple locations in the biological sample, thereby detecting the locations of the analytes associated with the complements of the barcode regions hybridized by the first library of intermediate probes; (e) detecting signals or absence thereof associated with the decoding regions of the first library of intermediate probes at at least a subset of the first multiple locations detected in step (d), in order to detect the first plurality of analytes; (f) contacting the biological sample with a second library of intermediate probes for a second plurality of analytes, wherein: each intermediate probe of the second library comprises (i) a hybridization region configured to hybridize to the complement of the barcode region of a primary probe, (ii) a second common spot-calling region, and (iii) a decoding region; (g) detecting signals associated with the second common spot-calling regions at second multiple locations in the biological sample, thereby detecting the locations of the analytes associated with the complements of the barcode regions hybridized by the second library of intermediate probes; and (h) detecting signals or absence thereof associated with the decoding regions of the second library of intermediate probes at at least a subset of the second multiple locations detected in step (g), in order to detect the second plurality of analytes.

In any of the embodiments herein, the first plurality of analytes and the second plurality of analytes do not need to comprise a common analyte, or can comprise one or more common analytes.

In any of the embodiments herein, the first multiple locations and the second multiple locations do not need to spatially overlap, or do not need to comprise more than 1%, more than 5%, more than 10%, more than 20%, or more than 30% spatially overlapping locations.

In any of the embodiments herein, the method can comprise aligning the signals associated with the spot-calling regions with the signals or absence thereof associated with the barcode regions or complements thereof. In any of the embodiments herein, the signals associated with the spot-calling regions can be used for registration of the signals or absence thereof associated with the barcode regions or complements thereof. In any of the embodiments herein, the method can comprise aligning the signals associated with the spot-calling regions with the signals or absence thereof associated with the decoding regions. In any of the embodiments herein, the signals associated with the spot-calling regions can be used for registration of the signals or absence thereof associated with the decoding regions. In any of the embodiments herein, the registration can comprise associating the signals or absence thereof associated with the decoding region or barcode regions or complements thereof with the locations of detected signals associated with the spot-calling regions.

In some embodiments, provided herein is a kit for analyzing a biological sample, comprising: (a) a plurality of primary probes, wherein: each primary probe comprises (i) a binding region configured to bind to an analyte, and (ii) a barcode region associated with the analyte; and (b) a library of intermediate probes, wherein: each intermediate probe comprises (i) a hybridization region configured to hybridize to the barcode region of a primary probe of the plurality of primary probes or a complement of the barcode region, (ii) a spot-calling region, and optionally (iii) a decoding region; and two or more of the intermediate probes in the library comprise a common spot-calling region. In any of the embodiments herein, the kit can comprise detectable probes that are configured to hybridize to the spot-calling region of the intermediate probe library. In any of the embodiments herein, each intermediate probe can comprise a decoding region; and the kit further can comprise detectable probes that are configured to hybridize to the decoding regions of the intermediate probes. In any of the embodiments herein, the detectable probes can be configured to hybridize to the spot-calling region and/or the detectable probes configured to hybridize to the decoding regions are fluorescently labeled and/or configured to directly or indirectly bind to fluorescently labeled probes.

### BRIEF DESCRIPTION OF THE DRAWINGS

The drawings illustrate certain features and advantages of this disclosure. These embodiments are not intended to limit the scope of the appended claims in any manner.

FIGS. 1A-1D show schematics illustrating exemplary intermediate probe libraries that can be used for spot-calling and/or decoding.

FIGS. 2A-2B show schematics illustrating exemplary intermediate probe libraries for sequential spot-calling and decoding cycles in a biological sample.

FIGS. 3A-3D show schematics illustrating an exemplary cycle of spot-calling and decoding using intermediate probes.

FIGS. 4A-4B show schematics illustrating examples of undiluted and diluted spot-calling in a biological sample with analytes that are crowded and/or overlapping.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Profiling of nucleic acids and/or other analytes in a biological sample, such as a cell or tissue sample, is essential for biomedical research and clinical applications. Microscopy imaging, which can resolve multiple analytes such as mRNAs in single cells, can provide valuable information regarding analyte abundance and localization in situ, which can be important for understanding the molecular basis of cell identity and developing treatment for diseases. In multiplex assays where multiple signals are detected simultaneously, it is important that individual signals are distinguished from one another so that as much information as possible can be collected from the assays. For example, in microscopy-based optical assays, individual “spots” emitting optical signals often need to be resolved from adjacent spots in a sample. However, resolving a large number of signals of varying strengths that are in close proximity remains challenging, and improved methods are needed.

In multiplex assays, such as multiplexed in situ gene expression and/or protein analysis, signal crowding problems can arise when there are a large number of signals to be detected. For example, combinatorial barcoding approaches are often used to encode a large number of analytes, and optical signals (e.g., spots in fluorescent microscopy) from the analytes or probes bound thereto are detected and decoded. Because the number of spots detected typically scales with the number of analytes assayed, a sample can become crowded with signal spots that overlap with each other thereby making resolution of individual spots difficult and reducing overall assay sensitivity. Thus, spatial overlap (e.g., optical crowding) may limit the ability to multiplex in assays such as microscopy-based nucleic acid hybridization or sequencing assays. In some cases, signal crowding may be especially problematic when detecting signals for spot-calling and using the spot-calling signals for image registration if crowded spots cannot be accurately resolved and called.

In some aspects, signal crowding may arise when one or more of the signals being detected are significantly stronger (e.g., have a significantly larger amplitude) than other signal(s). For example, in the same microscope field of view, one or more fluorescent spots may be significantly stronger than other spots including neighboring spots. In some aspects, signal crowding may also arise when one or more of the signals being detected are in close proximity (e.g., overlapping to some degree) to one another or with other signal(s), such as overlapping signals observed in the same microscope field of view. When a large number of signals (e.g., “spots”) are present or when the amplitude of a signal is significantly greater than that of another signal, it can be difficult to accurately and reliably detect all of the signals in the same field of view and/or in the same detection channel (e.g., the same fluorescent channel). In some examples, this can cause weaker (e.g., lower amplitude) or overlapping signals to be “crowded out” or masked, which ultimately leads to information from the system being lost. In such circumstances, the effective dynamic range of the detection assay can be reduced.

In certain methods of in situ analysis of gene expression, the identification of specific analytes in a sample includes two processes: (i) spot-calling (mapping or registering the locations of all transcripts of interest, e.g., “spots”), and (ii) decoding (obtaining fluorescent readouts to determine the identity of analytes in the locations mapped during spot-calling).

In some methods of spot-calling, primary probes bound to analytes, or products thereof (e.g. amplification products generated from primary probes), can comprise a common “universal” anchor sequence that corresponds to all target analytes. The anchor sequence is hybridized by a detectable (e.g. fluorescently labeled) oligonucleotide for spot-calling. The detectable oligonucleotide in turn can be used to generate fluorescent signals (e.g. spots) that are detected to map the locations of all analytes of interest in the sample. As the number of target analytes is increased, signal crowding or overlapping can make spot-calling difficult or impossible using a universal anchor sequence. Furthermore, signals associated with the anchor sequence can be crowded because the anchor sequence may be amplified, e.g., by rolling circle amplification (RCA), together with sequences (e.g., barcode sequences) used for decoding.

For decoding, each target analyte may be contacted and associated with a primary probe comprising a barcode region that corresponds to a specific analyte. In some embodiments, the barcode region (or a complement thereof) that is present in the primary probe or a product of the primary probe (e.g. a hybridization and/or amplification product, such as an RCA product) can be hybridized by detectable probes, which in turn can be used to generate fluorescent readouts for determining the identities of analytes in specific locations. In some methods, to generate the fluorescent readouts, an intermediate probe can be hybridized to the barcode region of a primary probe or to a product comprising a sequence of the primary probe or complement thereof. The intermediate probes can also comprise a decoding region that is directly or indirectly hybridized by a detectable probe.

In some aspects, provided herein are methods to optimize spot-calling and decoding schemes using intermediate probes and intermediate probe libraries that hybridize to primary probes or products thereof (e.g., RCA products), and that further comprise a spot-calling region. In some aspects, intermediate probes with spot-calling regions comprising common sequences across a plurality of analytes may provide certain benefits such as simplifying the assay protocol and reducing manufacturing costs. In some examples, common or universal sequences for spot-calling on the intermediate probes may provide benefits compared to using a universal anchor sequence present in the primary probes or products thereof for spot-calling.

In some aspects, provided herein are intermediate probes that comprise (i) a hybridization region that hybridizes to a barcode region associated with an analyte or complement thereof, and (ii) a spot-calling region, which can be directly or indirectly hybridized by detectable probes for spot-calling. In some embodiments, because spot-calling is performed using a sequence on the intermediate probe, as opposed to an anchor sequence comprised by a primary probe or product thereof (e.g., an RCA product generated using the primary probe and comprising complements of the anchor sequence), detectable probes for spot-calling can be more efficiently stripped and removed from the biological sample, for example when multiple sequential cycles of hybridization, detection, stripping, and removal are performed. In some instances, an intermediate probe described herein may remain hybridized to the primary probe (e.g., or a product thereof comprising a complement of the barcode region) for more than one cycle (e.g., the intermediate probe can be used for spot-calling and one or more rounds of decoding). In some aspects, the spot-calling region of the intermediate probe can also be independently designed and optimized for fluorescent labeling and stripping. In some embodiments, a sequence for spot-calling on the intermediate probe provided herein (as opposed to an anchor sequence comprised by a primary probe) can be easier to redesign and optimize to achieve the best performance in spot-calling, without affecting other aspects of the assay. For example, redesigning an anchor sequence in a primary probe could affect other aspects of the primary probe performance (e.g., sensitivity, specificity, etc.) and would require testing to evaluate any such changes. In contrast, in the methods provided herein, the spot-calling region is provided in the intermediate probe that hybridizes to the barcode region of the primary probe or product thereof, so the primary probes provided herein do not need to comprise a spot-calling region (e.g., a spot-calling sequence such as an anchor sequence). Thus, the spot-calling region can be redesigned and optimized without affecting the design and performance of the primary probe.

In some aspects, provided herein are different subsets of intermediate probes that comprise different spot-calling regions, allowing spot-calling to be performed for a smaller number of analytes at a time. In this way, orthogonal spot-calling regions on the intermediate probes are used to perform “diluted” spot-calling to avoid signal overcrowding. The approach enables spot-calling without significant overlap, or reduced overlap between spot-calling signals. Diluted spot-calling can be performed by generating different signals associated with the different spot-calling regions (e.g. using different fluorophores), or in sequential cycles in an iterative manner. Such diluted spot-calling provides an additional advantage over traditional methods of spot-calling using a universal anchor sequence to generate a single undiluted spot-calling signal.

In some aspects, the intermediate probes provided herein can further comprise a decoding region to be used for decoding, e.g., generating fluorescent readouts for determining the identities of analytes in specific locations. This allows spot-calling and decoding steps to be performed by generating signals from different regions of the same intermediate probe. Furthermore, stripping of the intermediate probe is sufficient for removing both signals for spot-calling and decoding.

Various signal amplification methods (e.g., as further described in Section VI) can be performed with the methods provided herein, using probes that directly or indirectly bind to specific analytes. Amplification methods can be enzymatic or non-enzymatic. For example, amplification can be achieved using detectable reactive molecules with probe hybridization, targeted assembly of branched structures (e.g., bDNA or branched assay using locked nucleic acid (LNA)), programmed in situ growth of concatemers by enzymatic rolling circle amplification (RCA), hybridization chain reaction (HCR), assembly of topologically catenated DNA structures using serial rounds of chemical ligation (clampFISH), and signal amplification via hairpin-mediated concatemerization. In some embodiments, amplification is performed using rolling circle amplification (RCA), and the RCA product comprises the barcode region or complement thereof that is hybridized by the intermediate probe.

In some embodiments, provided herein is a method for analyzing a biological sample, comprising: (a) contacting the biological sample with a plurality of primary probes, wherein: each primary probe comprises (i) a binding region configured to bind to a different analyte, and (ii) a barcode region associated with the analyte; (b) contacting the biological sample with a library of intermediate probes, wherein: each intermediate probe comprises (i) a hybridization region configured to hybridize to the barcode region of a primary probe of the plurality of primary probes or a complement of the barcode region, (ii) a spot-calling region, and optionally (iii) a decoding region; and two or more of the intermediate probes in the library comprise a common spot-calling region; (c) detecting signals associated with the spot-calling regions of the library of intermediate probes at multiple locations in the biological sample, thereby detecting the locations of the analytes associated with the barcode regions or complements thereof hybridized by the library of intermediate probes; and (d) detecting signals or absence thereof associated with the barcode regions or complements thereof at at least a subset of the locations detected in step (c).

Any of the methods disclosed herein may comprise one or more wash steps prior to or after any of the steps of the method. In some embodiments, the one or more wash steps comprise stringent wash to remove unbound and/or nonspecifically bound probe molecules from the biological sample.

In some embodiments, the hybridization region of the intermediate probe hybridizes to the barcode region of the primary probe. In some embodiments, the hybridization region of the intermediate probe hybridizes to the complement of the barcode region. The method may comprise a step of generating a product of the primary probe after contacting the biological sample with the plurality of primary probes, wherein the product comprises the barcode region of the primary probe or complement thereof. The product may be a rolling circle amplification (RCA) product comprising multiple copies of the barcode region or complement thereof.

The detecting of signals associated with the spot-calling regions of the library of intermediate probes may comprise detecting signals associated with detectable probes that are hybridized to the spot-calling regions in the library of intermediate probes. In some instances, the detectable probes hybridized to the spot-calling regions are fluorescently labeled and/or configured to directly or indirectly bind to fluorescently labeled probes. The detecting of signals associated with the barcode regions or complements thereof may comprise: detecting signals associated with detectable probes that are hybridized to the barcode regions or complements thereof; and/or detecting signals associated with detectable probes that are hybridized to intermediate probes which are in turn hybridized to the barcode regions or complements thereof. In some instances, the detectable probes hybridized to the barcode regions or complements thereof and/or the detectable probes hybridized to the intermediate probes which are in turn hybridized to the barcode regions or complements thereof are fluorescently labeled and/or configured to directly or indirectly bind to fluorescently labeled probes. In some embodiments, the method comprises aligning the signals associated with the spot-calling regions with the signals or absence thereof associated with the barcode regions or complements thereof.

In some embodiments, all of the intermediate probes in the library comprise the same common spot-calling region. In some embodiments, the library of intermediate probes comprises a first sub-library and a second sub-library, the intermediate probes in the first sub-library comprise a first common spot-calling region, the intermediate probes in the second sub-library comprise a second common spot-calling region, and the first and second common spot-calling regions are different. In some embodiments, the detecting of signals associated with the spot-calling regions may comprise: (i) detecting signals associated with the first common spot-calling region of the first sub-library of intermediate probes at multiple locations in the biological sample, thereby detecting the locations of the analytes associated with the barcode regions or complements thereof hybridized by the first sub-library of intermediate probes; and/or (ii) detecting signals associated with the second common spot-calling region of the second sub-library of intermediate probes at multiple locations in the biological sample, thereby detecting the locations of the analytes associated with the barcode regions or complements thereof hybridized by the second sub-library of intermediate probes. In some embodiments, the detecting of signals associated with the spot-calling regions may comprise: contacting the biological sample with detectable probes that hybridize to the first common spot-calling region in the first sub-library of intermediate probes; and/or contacting the biological sample with detectable probes that hybridize to the second common spot-calling region in the second sub-library of intermediate probes. In some embodiments, the detecting of signals associated with the barcode regions or complements thereof comprises: detecting signals associated with the barcode regions or complements thereof at at least a subset of the locations associated with the first common spot-calling region of the first sub-library of intermediate probes; and/or detecting signals associated with the barcode regions or complements thereof at at least a subset of the locations associated with the second common spot-calling region of the second sub-library of intermediate probes. In some embodiments, the first sub-library comprises intermediate probes configured to hybridize to the barcode regions of primary probes or complements thereof associated with two or more different analytes; and/or the second sub-library comprises intermediate probes configured to hybridize to the barcode regions of primary probes or complements thereof associated with two or more different analytes. In some embodiments, one or more intermediate probes in the first sub-library and one or more intermediate probes in the second sub-library are configured to hybridize to the barcode regions of primary probes or complements thereof associated with different analytes.

In some embodiments, the method further comprises contacting the biological sample with a library of decoding intermediate probes, wherein each decoding intermediate probe comprises (i) a hybridization region configured to hybridize to the barcode region of a primary probe of the plurality of primary probes or a complement of the barcode region, and (ii) a decoding region. The biological sample may be further contacted with detectable probes that hybridize to the decoding regions of the library of decoding intermediate probes. The method may comprise detecting signals or absence thereof associated with the detectable probes. In some embodiments, the detectable probes hybridized to the decoding regions of the library of decoding intermediate probes are fluorescently labeled and/or configured to directly or indirectly bind to fluorescently labeled probes.

The method may further comprise contacting the biological sample with a subsequent library of decoding intermediate probes, wherein each subsequent decoding intermediate probe comprises (i) a hybridization region configured to hybridize to the barcode region of a primary probe of the plurality of primary probes or a complement of the barcode region, and (ii) a subsequent decoding region. The biological sample may be contacted with subsequent detectable probes that hybridize to the subsequent decoding regions of the subsequent library of decoding intermediate probes, and detecting signals or absence thereof associated with the subsequent detectable probes hybridized to the subsequent decoding regions. The decoding intermediate probes and the subsequent decoding intermediate probes may hybridize to the same barcode sequence (or complement thereof) or different barcode sequences (or complements thereof) of the barcode region. The different barcode sequences can be partially overlapping. The decoding region and the subsequent decoding region may be configured to hybridize to the same detectable probe. In some embodiments, the decoding region and the subsequent decoding region are identical in sequence and hybridize to detectable probes comprising the same detectable label. The decoding region and the subsequent decoding region may be configured to hybridize to detectable probes of different sequences. In some embodiments, each detectable probe of a different sequence comprises a different detectable label.

In some of any of the provided embodiments, the intermediate probes in the library of intermediate probes in (b) comprise decoding regions. In such embodiments, the method may comprise contacting the biological sample with detectable probes that hybridize to the decoding regions of the library of intermediate probes. The method may comprise detecting signals or absence thereof associated with the detectable probes. In some embodiments, the detectable probes are fluorescently labeled and/or configured to directly or indirectly bind to fluorescently labeled probes.

In some embodiments, the method comprises generating a signal code sequence for one or more of the multiple locations, the signal code sequence comprising signal codes corresponding to: the signals (or absence thereof) associated with the detectable probes hybridized to the decoding regions of the library of intermediate probes, the signals (or absence thereof) associated with the detectable probes hybridized to the decoding regions of the library of decoding intermediate probes, and/or the signals (or absence thereof) associated with the subsequent detectable probes hybridized to the decoding regions of the library of subsequent decoding intermediate probes, wherein the signal code sequence corresponds to an analyte, thereby detecting the analyte at the one or more of the multiple locations.

The barcode region of a primary probe may comprise one or more barcode sequence or a combination of barcode sequences. In some aspects, the barcode sequence or the combination of barcode sequences corresponds to the analyte associated with the barcode region.

Each primary probe may be independently selected from the group consisting of: a primary probe comprising a 3′ or 5′ overhang, optionally wherein the 3′ or 5′ overhang comprises one or more barcode sequences; a primary probe comprising a 3′ overhang and a 5′ overhang, optionally wherein the 3′ overhang and the 5′ overhang each independently comprises one or more barcode sequences; a circular primary probe; and a circularizable primary probe or probe set. Each intermediate probe may be independently selected from the group consisting of: an intermediate primary probe comprising a 3′ or 5′ overhang, optionally wherein the 3′ or 5′ overhang comprises one or more spot-calling regions and/or decoding regions; an intermediate probe comprising a 3′ overhang and a 5′ overhang, optionally wherein the 3′ overhang and the 5′ overhang each independently comprise one or more spot-calling regions and/or decoding regions; a circular intermediate probe; and a circularizable intermediate probe or probe set.

In some embodiments, an analyte comprises a nucleic acid, a protein, a carbohydrate, a lipid, a small molecule, or a complex of any of the foregoing. In some embodiments, the analyte is a genomic DNA, cDNA, or mRNA. In some embodiments, the analyte is a protein, and the primary probe comprises a binding region and one or more reporter oligonucleotides conjugated thereto, wherein the binding region binds the protein and the one or more reporter oligonucleotides comprise the barcode region.

In some embodiments, the biological sample is non-homogenized and optionally selected from the group consisting of a formalin-fixed, paraffin-embedded (FFPE) sample, a frozen tissue sample, and a fresh tissue sample. The biological sample may be fixed or not fixed. In some embodiments, the biological sample is permeabilized. In some embodiments, the biological sample is embedded in a matrix. In some embodiments, the matrix comprises a hydrogel. In some embodiments, the biological sample is cleared. In some embodiments, the clearing comprises contacting the biological sample with a proteinase. In some embodiments, the biological sample is crosslinked. The biological sample may be a tissue slice between about 1 μm and about 50 μm in thickness. In some embodiments, the tissue slice is between about 5 μm and about 35 μm in thickness.

In some embodiments, disclosed herein is a method for analyzing a biological sample, comprising: (a) contacting the biological sample with a plurality of primary probes, wherein: each primary probe comprises (i) a binding region configured to bind to a different analyte, and (ii) a barcode region associated with the analyte; and each primary probe is circular or circularizable; (b) generating rolling circle amplification (RCA) products of the primary probes, wherein each RCA product comprises a complement of the barcode region of the primary probe; (c) contacting the biological sample with a library of intermediate probes, wherein: each intermediate probe comprises (i) a hybridization region configured to hybridize to the complement of the barcode region of a primary probe, (ii) a spot-calling region, and (iii) a decoding region; and two or more of the intermediate probes in the library comprise a common spot-calling region; (d) detecting signals associated with the spot-calling regions of the library of intermediate probes at multiple locations in the biological sample, thereby detecting the locations of the analytes associated with the complements of the barcode regions hybridized by the library of intermediate probes; and (e) detecting signals or absence thereof associated with the decoding regions of the library of intermediate probes at at least a subset of the locations detected in step (d).

In some embodiments, a unique signal code sequence is assigned to each analyte to be analyzed; each signal code sequence is derivable by interrogating the complements of the barcode regions with sequential intermediate probe libraries; the method further comprises one or more sequential cycles, each cycle comprising: (i) contacting the biological sample with a subsequent intermediate probe library, wherein each intermediate probe in the subsequent intermediate probe library comprises (a) a hybridization region configured to hybridize to the complement of the barcode region of a primary probe, (b) a decoding region, and optionally (c) a spot-calling region; (ii) optionally detecting signals associated with the spot-calling region of the subsequent intermediate probe library at multiple locations in the biological sample, thereby detecting the locations of the analytes associated with the complements of the barcode regions hybridized by the subsequent intermediate probe library; and (iii) detecting signals or absence thereof associated with the decoding regions of intermediate probes in the subsequent intermediate probe library at at least a subset of the locations detected in step (c) and/or step (ii); a sequence of signals sequentially detected at at least a subset of the multiple locations in the biological sample in the sequential cycles delineates a signal code sequence corresponding to a particular analyte, thereby revealing the location of the analyte in the biological sample; and the locations of one or more of the analytes in the biological sample are determined.

In some aspects, disclosed herein is a method for analyzing a biological sample, comprising: (a) contacting the biological sample with a plurality of primary probes, wherein: each primary probe comprises (i) a binding region configured to bind to a different analyte, and (ii) a barcode region associated with the analyte; and each primary probe is circular or circularizable; (b) generating rolling circle amplification (RCA) products of the primary probes, wherein each RCA product comprises a complement of the barcode region of the primary probe; (c) contacting the biological sample with a first library of intermediate probes for a first plurality of analytes, wherein: each intermediate probe of the first library comprises (i) a hybridization region configured to hybridize to the complement of the barcode region of a primary probe, (ii) a first common spot-calling region, and (iii) a decoding region; (d) detecting signals associated with the first common spot-calling regions at first multiple locations in the biological sample, thereby detecting the locations of the analytes associated with the complements of the barcode regions hybridized by the first library of intermediate probes; (e) detecting signals or absence thereof associated with the decoding regions of the first library of intermediate probes at at least a subset of the first multiple locations detected in step (d), in order to detect the first plurality of analytes; (f) contacting the biological sample with a second library of intermediate probes for a second plurality of analytes, wherein: each intermediate probe of the second library comprises (i) a hybridization region configured to hybridize to the complement of the barcode region of a primary probe, (ii) a second common spot-calling region, and (iii) a decoding region; (g) detecting signals associated with the second common spot-calling regions at second multiple locations in the biological sample, thereby detecting the locations of the analytes associated with the complements of the barcode regions hybridized by the second library of intermediate probes; and (h) detecting signals or absence thereof associated with the decoding regions of the second library of intermediate probes at at least a subset of the second multiple locations detected in step (g), in order to detect the second plurality of analytes. In some embodiments, the first plurality of analytes and the second plurality of analytes do not comprise a common analyte or comprise one or more common analytes. In some embodiments, the first multiple locations and the second multiple locations do not spatially overlap or comprise no more than 1%, no more than 5%, no more than 10%, no more than 20%, or no more than 30% spatially overlapping locations.

In some aspects, provided herein is a kit for analyzing a biological sample, comprising: (a) a plurality of primary probes, wherein: each primary probe comprises (i) a binding region configured to bind to a different analyte, and (ii) a barcode region associated with the analyte; and (b) a library of intermediate probes, wherein: each intermediate probe comprises (i) a hybridization region configured to hybridize to the barcode region of a primary probe of the plurality of primary probes or a complement of the barcode region, (ii) a spot-calling region, and optionally (iii) a decoding region; and two or more of the intermediate probes in the library comprise a common spot-calling region. In some embodiments, each intermediate probe comprises a decoding region. In some embodiments, the kit further comprises detectable probes that are configured to hybridize to the spot-calling region of the intermediate probe library. In some embodiments, the kit further comprises detectable probes that are configured to hybridize to the decoding regions of the intermediate probes. In some embodiments, the detectable probes configured to hybridize to the spot-calling region and/or the detectable probes configured to hybridize to the decoding regions are fluorescently labeled and/or configured to directly or indirectly bind to fluorescently labeled probes.

In some embodiments of the method, a unique signal code sequence is assigned to each analyte to be analyzed; each signal code sequence is derivable by interrogating the barcode regions or products thereof with sequential probe libraries; the method further comprises one or more sequential cycles, each cycle comprising: (a) contacting the biological sample with a new probe library, wherein each probe in the new probe library comprises (i) a hybridization region capable of hybridizing to the barcode region of a primary probe or a product thereof, and (ii) a decoding region; (b) detecting signals associated with the decoding regions of probes in the new probe library at multiple locations in the biological sample; a sequence of signals sequentially detected at at least a subset of the multiple locations in the biological sample in the one or more sequential cycles delineates a signal code sequence corresponding to a particular analyte, thereby revealing the location of the analyte in the biological sample; and the locations of one or more of the analytes in the biological sample are determined.

### II. Samples, Analytes, and Target Sequences

A. Samples

A sample disclosed herein can be or is derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., one or more probes such as a primary probe, a circularizable or circularized probe or probe set (e.g. a padlock probe), a circular probe, or an intermediate probe as described herein. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe (e.g., a padlock probe).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Any suitable method of non-chemical permeabilization can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each analyte (e.g. RNA or cDNA) analyte are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any suitable method (e.g., using streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and/or alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a circularizable probe such as a padlock probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly binds (e.g. hybridizes) to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example by using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (e.g. coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, the content of which is herein incorporated by reference in its entirety. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed. In some embodiments, the product is generated using a primary probe that binds directly or indirectly to an analyte in the biological sample.

(a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or a probe contacted with the sample (e.g., a primary probe). Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, and a PLISH (Proximity Ligation in situ Hybridization) probe set. The specific probe or probe set design can vary.

(b) Ligation

In some embodiments, a product of an endogenous analyte and/or a labeling agent is a ligation product. In some embodiments, the ligation product is formed between two or more endogenous analytes. In some embodiments, the ligation product is formed between an endogenous analyte and a labeling agent. In some embodiments, the ligation product is formed between two or more labeling agent. In some embodiments, the ligation product is an intramolecular ligation of an endogenous analyte. In some embodiments, the ligation product is an intramolecular ligation of a probe, for example, the circularization of a circularizable probe (e.g. a padlock probe) or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set (e.g., comprising the primary probe) capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead ligation occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe (e.g. padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, such as steps comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labeling agent, a probe or probe set bound to the analyte (e.g., a primary probe such as a circularizable probe bound to genomic DNA, mRNA, or cDNA), or a probe or probe set bound to the labeling agent (e.g., a primary probe such as a circularizable probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and are used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some embodiments, in a primer extension reaction, two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) can include linear RCA, branched RCA, dendritic RCA, or any combination thereof. See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; and U.S. Pat. Nos. 6,054,274, 6,291,187, 6,323,009, 6,344,329 and 6,368,801, each of which are herein incorporated by reference in their entireties). Exemplary polymerases for use in RCA include DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary modified nucleotides include amine-modified nucleotides. In some aspects of the methods, for example, modified nucleotides can be incorporated for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modifications and polymer matrixes that can be employed in accordance with the provided embodiments include those described in, for example, US 2016/0024555, US 2018/0251833, and US 2017/0219465, the entire contents of each of which are incorporated herein by reference. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. Various assays can be used for the detection of numerous different analytes, which use an RCA-based detection system, e.g., where the signal is provided by generating an RCA product (RCP) from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

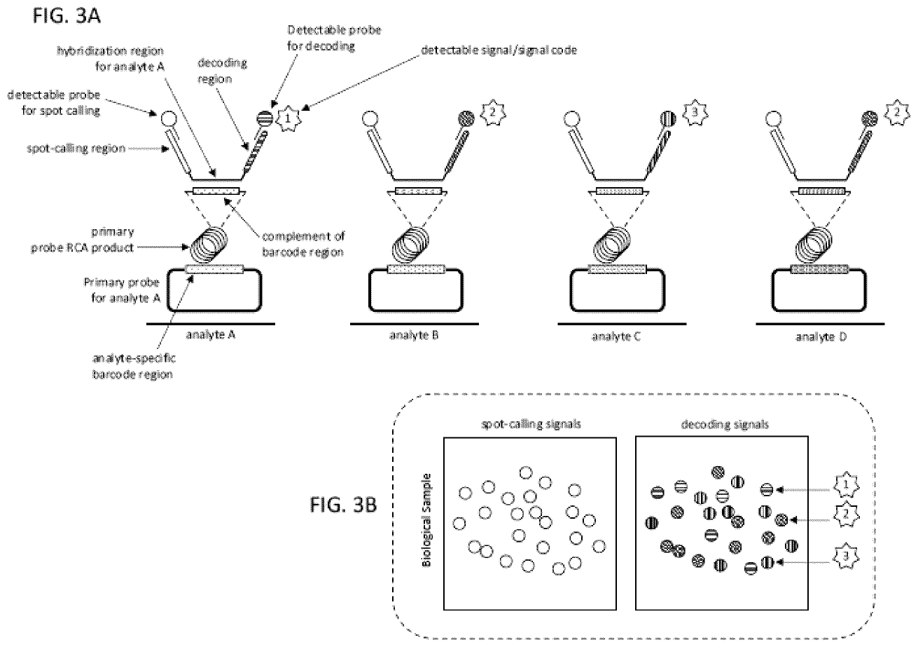

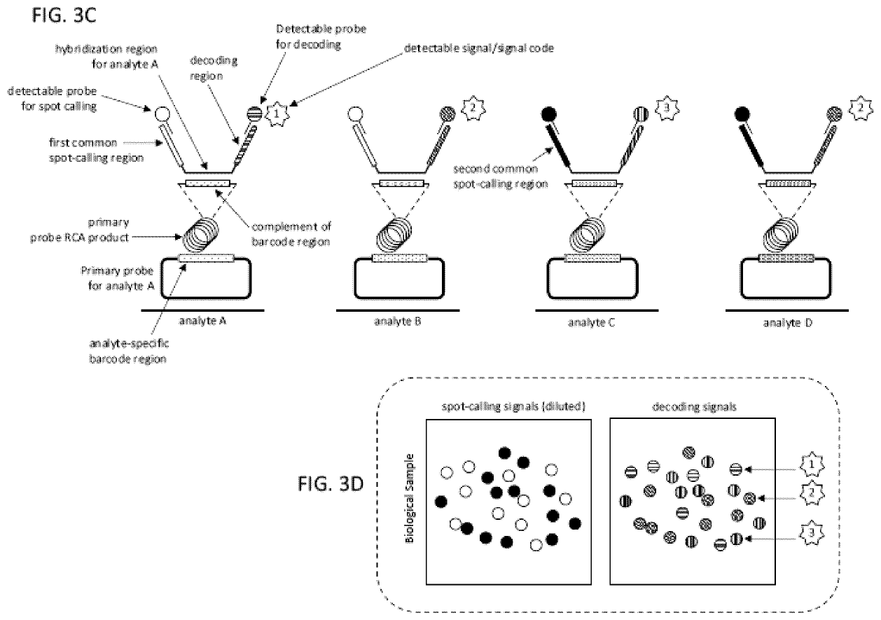

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g. as shown in FIG. 3A) may be a hybridization complex formed of a cellular nucleic acid (e.g., analyte) in a sample and an exogenously added nucleic acid probe (e.g., primary probes). In other examples, a product comprising a sequence for hybridizing to the hybridization region of a probe disclosed herein (e.g., the complement of the analyte-specific barcode region present in the RCA product of the primary probe as shown in FIG. 3A) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a sequence for hybridizing to the hybridization region of a probe disclosed herein (e.g., the intermediate probes as shown in FIG. 3A) may be a probe hybridizing to an RCP. The probe (e.g., intermediate probe) may comprise an overhang that does not hybridize to the RCP but hybridizes to another probe (e.g., the spot-calling region or decoding region of intermediate probes as shown in FIG. 3A).

### III. Probes and Products Thereof

Disclosed herein in some aspects are nucleic acid probes and/or probe sets that are introduced into a cell or used to otherwise contact a biological sample such as a tissue sample. The probes (e.g., the primary probe, intermediate probe disclosed herein, and/or any detectable probe disclosed herein) may comprise any of a variety of entities that can hybridize to a nucleic acid, typically by Watson-Crick base pairing, such as DNA, RNA, LNA, PNA, etc. The nucleic acid probe may comprise a targeting sequence that is able to directly or indirectly bind to at least a portion of a target nucleic acid. For example, the nucleic acid probe may hybridize to the target nucleic acid, or the nucleic acid probe may hybridize to another probe or hybridization complex that is in turn hybridized to the target nucleic acid. The nucleic acid probe may be able to bind to a specific target nucleic acid (e.g., an mRNA, or other nucleic acids disclosed herein). In some embodiments, the nucleic acid probes may be detected using a detectable label, and/or by using intermediate nucleic acid probes able to bind to the nucleic acid probes, which are themselves detectable. In some embodiments, the nucleic acid probes (e.g., primary probes and/or intermediate probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

In some embodiments, more than one type of primary nucleic acid probes may be contacted with a sample, e.g., simultaneously or sequentially in any suitable order, such as in sequential probe hybridization/unhybridization cycles. In some embodiments, more than one type of intermediate nucleic acid probes may be contacted with a sample, e.g., simultaneously or sequentially in any suitable order, such as in sequential probe hybridization/unhybridization cycles. In some embodiments, the intermediate probes may comprise probes that bind to a product of a primary probe targeting an analyte. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes may be contacted with a sample, e.g., simultaneously or sequentially in any suitable order, such as in sequential probe hybridization/unhybridization cycles. In some embodiments, the detectably labeled probes (e.g. detectable probes and/or fluorescently labeled probes) may comprise probes that bind to one or more primary probes, one or more intermediate probes, one or more higher order probes, one or more intermediate probes between a primary/second/higher order probes, and/or one or more detectably or non-detectably labeled probes (e.g., as in the case of a hybridization chain reaction (HCR), a branched DNA reaction (bDNA), or the like). In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, intermediate, higher order probes, and/or detectably labeled probes) can be contacted with a sample, e.g., simultaneously or sequentially in any suitable order. Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the targeting region/sequence, the recognition region/sequence, or the hybridization region/sequence) of a probe (e.g. the hybridization region of the intermediate probes of the library of intermediate probes described herein) may be positioned anywhere within the probe. For instance, the target-binding sequence in the binding region of a primary probe that binds to a target nucleic acid can be 5′ or 3′ to any barcode sequence in the primary probe. Likewise, the target-binding sequence in the hybridization region of an intermediate probe (which binds to a primary probe or complement or product thereof) can be 5′ or 3′ to any barcode sequence in the intermediate probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence in the binding region of a primary nucleic acid probe may be determined with reference to a target nucleic acid (e.g., a cellular RNA or a reporter oligonucleotide of a labeling agent for a cellular analyte) that is present or suspected of being present in a sample. In some embodiments, more than one target-binding sequence can be used to identify a particular analyte comprising or associated with a target nucleic acid. The more than one target-binding sequence can be in the same probe or in different probes. For instance, multiple probes can be used, sequentially and/or simultaneously, that can bind to (e.g., hybridize to) different regions of the same target nucleic acid. In other examples, a probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

In some embodiments, a primary probe disclosed herein can comprise a padlock-like probe or probe set, such as one described in US 2019/0055594, US 2021/0164039, US 2016/0108458, or US 2020/0224243, each of which is incorporated herein by reference in its entirety. Any suitable combination of the probe designs described herein can be used.

In some embodiments, a primary probe disclosed herein can comprise two or more parts, for example, being similar to: a split FISH probe or probe set described in WO 2021/167526A1 or Goh et al., “Highly specific multiplexed RNA imaging in tissues with split-FISH,” Nat Methods 17(7):689-693 (2020), which are incorporated herein by reference in their entireties; a Z-probe or probe set, such as one described in U.S. Pat. No. 7,709,198 B2, U.S. Pat. No. 8,604,182 B2, U.S. Pat. No. 8,951,726 B2, U.S. Pat. No. 8,658,361 B2, or Tripathi et al., “Z Probe, An Efficient Tool for Characterizing Long Non-Coding RNA in FFPE Tissues,” Noncoding RNA 4(3):20 (2018), which are incorporated herein by reference in their entireties; an HCR initiator or amplifier, such as one described in U.S. Pat. No. 7,632,641 B2, US 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), all of which are incorporated herein by reference in their entireties; a PLAYR probe or probe set, such as one described in US 2016/0108458 A1 or Frei et al., “Highly multiplexed simultaneous detection of RNAs and proteins in single cells,” Nat Methods 13(3):269-75 (2016), all of which are incorporated herein by reference in their entireties; a PUSH probe or probe set, such as one described in US 2020/0224243 A1 or Nagendran et al., “Automated cell-type classification in intact tissues by single-cell molecular profiling,” eLife 7:e30510 (2018), all of which are incorporated herein by reference in their entireties; a RollFISH probe or probe set such as one described in Wu et al., “RollFISH achieves robust quantification of single-molecule RNA biomarkers in paraffin-embedded tumor tissue samples,” Commun Biol 1, 209 (2018), which is hereby incorporated by reference in its entirety; a MERFISH probe or probe set, such as one described in US 2022/0064697 or Chen et al., “Spatially resolved, highly multiplexed RNA profiling in single cells,” Science 348(6233):aaa6090 (2015), all of which are incorporated herein by reference in their entireties; or a primer exchange reaction (PER) probe or probe set, such as one described in US 2019/0106733 A1, which is hereby incorporated by reference in its entirety. In some embodiments, a primary probe disclosed herein can comprise a probe that is ligated to itself or another probe using DNA-templated and/or RNA-templated ligation.

Any suitable circularizable probe or probe set, or indeed more generally circularizable reporter molecules, may be used to generate the RCA template which is used to generate the RCA product. In some embodiments, a circularizable probe or reporter is in the form of a linear molecule having ligatable ends which may be ligated to one another form a circularized molecule (e.g. to serve as the RCA template). The ends may be ligated together directly or indirectly, e.g. to each other, or to the respective ends of an intervening (“gap”) oligonucleotide or to an extended 3′ end of the circularizable probe. A circularizable probe may also be provided in two or more parts, namely two or more molecules (e.g. oligonucleotides) which may be ligated together to form a circle. Ligation may be templated using a ligation template. The circularizable probe (or template part or portion) may comprise at its respective 3′ and 5′ ends regions of complementarity to corresponding cognate complementary regions (or binding sites) in the ligation template, which may be adjacent to where the ends are directly ligated to each other, or non-adjacent, with an intervening “gap” sequence, where indirect ligation is to take place.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

In some aspects, the methods provided herein comprise performing rolling circle amplification of a circular probe or a circularized probe generated from a circularizable probe or probe set.

In some embodiments, a probe disclosed herein (e.g. a probe hybridized to the target nucleic acid) can comprise a 5′ flap which may be recognized by a structure-specific cleavage enzyme, e.g. an enzyme capable of recognizing the junction between a single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to a target, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/0224244 (each of which is incorporated herein by reference in its entirety) and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 (the content of which is herein incorporated by reference in its entirety) for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a primary nucleic acid probe can bind to a target nucleic acid in the sample, and an intermediate probe can be introduced to bind to the primary nucleic acid probe, where the intermediate probe or a product thereof can then be detected using detectably labeled probes (e.g., detectable probes). Higher order probes that directly or indirectly bind to the intermediate probe or product thereof may also be used, and the higher order probes or products thereof can then be detected using detectably labeled probes (e.g., detectable probes).

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the primary probes, intermediate probes, higher order probes, and/or detectably labeled probes may comprise any of a variety of entities able to hybridize a nucleic acid, e.g., DNA, RNA, LNA, and/or PNA, etc., depending on the application.

In some embodiments, the nucleic acid probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe disclosed herein lacks a detectable label. While a detectable label may be incorporated into an amplification product of a probe, such as via incorporation of a modified nucleotide into an RCA product of a circularized probe, the amplification product itself in some embodiments is not detectably labeled. In some embodiments, a probe that binds to the primary nucleic acid probe or a product thereof (e.g., an intermediate probe that binds to a barcode sequence or complement thereof in the primary nucleic acid probe or product thereof) comprises a detectable label and may be used to detect the primary nucleic acid probe or product thereof. In some embodiments, an intermediate probe disclosed herein lacks a detectable label, and a detectably labeled probe that binds to the intermediate probe or a product thereof (e.g., at a barcode sequence or complement thereof in the intermediate probe or product thereof) can be used to detect the intermediate probe. In some embodiments, signals associated with the detectably labeled probes can be used to detect one or more barcode sequences in the intermediate probe and/or one or more barcode sequences in the primary probe, e.g., by using sequential hybridization of detectably labeled probes. In some embodiments, the barcode sequences (e.g., in the intermediate probe and/or in the primary probe) are used to combinatorially encode a plurality of analytes of interest. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order.

In some embodiments, provided herein are probes, probe sets, and assay methods to couple target nucleic acid detection, signal amplification (e.g., through nucleic acid amplification such as RCA, and/or hybridization of a plurality of detectably labeled probes, such as in hybridization chain reactions and the like), and decoding of the barcodes.

In some aspects, a primary probe, an intermediate probe, and/or a higher order probe can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, a primary probe, an intermediate probe, and/or a higher order probe disclosed herein can comprise a circularizable probe, a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504, both of which are incorporated herein by reference in their entireties, and probes for Proximity Ligation Assay (PLA) for the simultaneous detection and quantification of nucleic acid molecules and protein-protein interactions), or any suitable combination thereof. In some embodiments, a primary probe, an intermediate probe, and/or a higher order probe disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template.

A. Primary Probes and Barcodes

Provided herein are methods comprising contacting a biological sample with a primary probe configured to bind to a target sequence which may be comprised in any analyte disclose herein, e.g., as described in Section II. In some aspects, the primary probe comprises i) a binding region configured to bind to an analyte and ii) a barcode region associated with the analyte (e.g., analyte-specific barcode region as shown in FIG. 3A and FIG. 3C). In some aspects, the primary probe is used to generate a product (e.g., amplification product) comprising multiple copies of a sequence that is a complement of the barcode region. In some embodiments, the primary probe is used to generate a product, wherein the product comprises the barcode region of the primary probe or complement thereof. In some aspects, provided herein is a method for analyzing a biological sample comprising contacting the biological sample with a plurality of primary probes. In some embodiments, each primary probe of the plurality of primary probes comprises (i) a binding region configured to bind to an analyte, and (ii) a barcode region associated with the analyte.

In some aspects, the primary probe includes a barcode region comprising one or more barcode(s), e.g., at least two, three, four, five, six, seven, eight, nine, ten, or more barcodes. In some aspects, barcodes can be used to spatially resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, a nucleic acid probe, such as a primary or an intermediate probe, may also comprise 1, 2, 3, 4, 5, 6, 7, 8, 9, 10, 11, 12, 13, 14, 15 or more, 20 or more, 32 or more, 40 or more, or 50 or more barcode sequences. As an illustrative example, a first probe may contain a first target-binding sequence, a first barcode sequence, and a second barcode sequence, while a second, different probe may contain a second target-binding sequence (that is different from the first target-binding sequence in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some embodiments, the methods provided herein can include analyzing the barcodes by sequential hybridization and detection with a plurality of detectable probes. In some embodiments, the detection is in situ. In some embodiments, the in situ detection herein can comprise sequential hybridization, e.g., sequencing by hybridization and/or sequential in situ fluorescence hybridization, for instance, for detecting a sequence of a barcode sequence (e.g., in an RCA product). Sequential fluorescence hybridization can involve sequential hybridization of detectable probes comprising an oligonucleotide and a detectable label. In some embodiments, a method disclosed herein comprises sequential hybridization of the probes disclosed herein, including detectably labeled probes (e.g., fluorophore conjugated oligonucleotides that can be used as detectable probes for spot-calling or for decoding) and/or probes (e.g., intermediate probes disclosed herein) that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference in their entireties.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences (or complements thereof) contained in the probes or RCA products are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 and US 2021/0164039, each of which are hereby incorporated by reference in their entirety.

B. Intermediate Probes

Provided herein are intermediate probes. In some embodiments, provided herein are libraries of intermediate probes. In some embodiments, provided herein is a method for analyzing a biological sample, wherein the method comprises contacting the biological sample with a library of intermediate probes. In some embodiments, each intermediate probe comprises (i) a hybridization region configured to hybridize to the barcode region of a primary probe of the plurality of primary probes or a complement of the barcode region, and (ii) a spot-calling region. In some embodiments, each intermediate probe further comprises (iii) a decoding region. In some embodiments, provided herein are decoding intermediate probes and libraries thereof that comprise (i) a hybridization region configured to hybridize to the barcode region of a primary probe of the plurality of primary probes or a complement of the barcode region, and (ii) a decoding region. The intermediate probes can be used in connection with any of the methods as described herein.

An intermediate probe disclosed herein may contain a recognition sequence (e.g., in the hybridization region) able to bind to or hybridize with a primary nucleic acid probe or a product thereof, e.g., at a barcode region or barcode sequence, or portion(s) thereof of the primary nucleic acid probe, or complement thereof in an RCA product of a primary probe. In some embodiments, an intermediate probe may bind to a combination of barcode sequences (which may be continuous or spaced from one another) in a primary nucleic acid probe, a product thereof, or a combination of primary nucleic acid probes. In some embodiments, the binding is specific, or the binding may be such that a recognition sequence preferentially binds to or hybridizes with only one of the barcode sequences or complements thereof that are present. The intermediate probe may also contain one or more detectable labels. If more than one intermediate probe is used, the detectable labels may be the same or different.

The recognition sequences (e.g., in the hybridization region) may be of any length, and multiple recognition sequences in the same or different intermediate nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In some embodiments, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

The intermediate probe may be provided as one oligonucleotide or two or more oligonucleotides. The recognition sequence (e.g., in the hybridization region) of the intermediate probe may be a contiguous nucleic acid sequence, or may be a split sequence comprising two or more nucleic acid sequences. Upon hybridization to the primary probe or product thereof, two adjacent nucleic acid sequences in the recognition sequence can be separated by a nick or a gap of one or more nucleotides. In some embodiments, the intermediate probe is provided as one oligonucleotide, and the recognition sequence is a contiguous nucleic acid sequence at the 3′ end, at the 5′ end, or between the 3′ and the 5′ ends of the intermediate probe. In some embodiments, the 3′ and 5′ ends of the intermediate probe can be ligated to each other using a splint. In some embodiments, the intermediate probe is provided as one oligonucleotide that is circularizable (but does not need to be circularized) upon hybridization to the primary probe or product thereof, and the recognition sequence is a split sequence comprising a sequence at the 3′ end of the intermediate probe and a sequence at the 5′ end of the intermediate probe. In some embodiments, the intermediate probe is provided as two oligonucleotides, and the recognition sequence comprises a first sequence on the 3′ end of one of the two oligonucleotides and a second sequence on the 5′ end of the other oligonucleotide. The two oligonucleotides may be ligated to each other using the primary probe or product thereof (e.g., a barcode sequence in the primary probe or product thereof) as template, with or without gapfilling prior to the ligation. The two oligonucleotides may additionally be ligated to each other using a splint that hybridizes to the 3′ end of one of the two oligonucleotides and the 5′ end of the other oligonucleotide.

The spot-calling region of an intermediate probe may be of any length, and multiple spot-calling regions in the same or different intermediate nucleic acid probes may be of the same or different lengths. If more than one spot-calling region is used, the spot-calling regions may independently have the same or different lengths. For instance, the spot-calling region may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the spot-calling region may be no more than 48, no more than 40, no more than 32, no more than 24, no more than 16, no more than 12, no more than 10, no more than 8, or no more than 6 nucleotides in length. Combinations of any of these are also possible, e.g., the spot-calling region may have a length of between 5 and 8, between 6 and 12, or between 7 and 15 nucleotides, etc. The spot-calling region may be located anywhere on the intermediate probe relative to the other components of the intermediate probe, e.g. the hybridization region or decoding region. The spot-calling region may be located on an overhang of the intermediate probe, e.g., a region that does not hybridize to the primary probe or product thereof. The spot-calling region may be positioned at the 3′ end or 5′ end of the intermediate probe. The spot-calling region may be 5′ or 3′ to the hybridization region. The spot-calling region and the decoding region can be on the same overhang (5′ or 3′ to the hybridization region) or on different overhangs of the intermediate probe. In some embodiments, the spot-calling region is 5′ to the decoding region. In some embodiments, the spot-calling region is 3′ to the decoding region. The spot-calling region may be a contiguous region in nucleic acid sequence, or may be a split region comprising two or more nucleic acid sequences that are not directly linked by a phosphodiester bond (e.g., they can be separated by one or more nucleotide residues). In some embodiments, the spot-calling region may comprise: i) a sequence on a 3′ overhang (e.g., at the 3′ end) of the intermediate probe and a sequence on a 5′ overhang (e.g., at the 5′ end) of the intermediate probe; ii) two sequences on the 3′ overhang which are separately by one or more nucleotide residues; iii) two sequences on the 5′ overhang which are separately by one or more nucleotide residues, or a combination thereof.

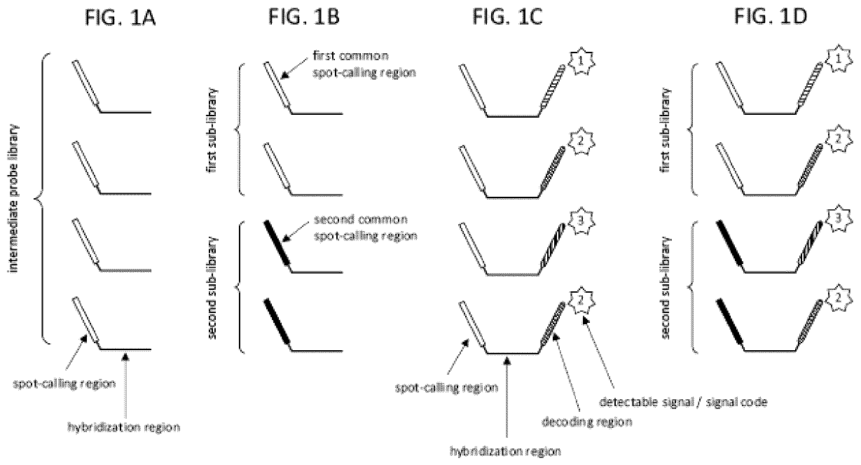

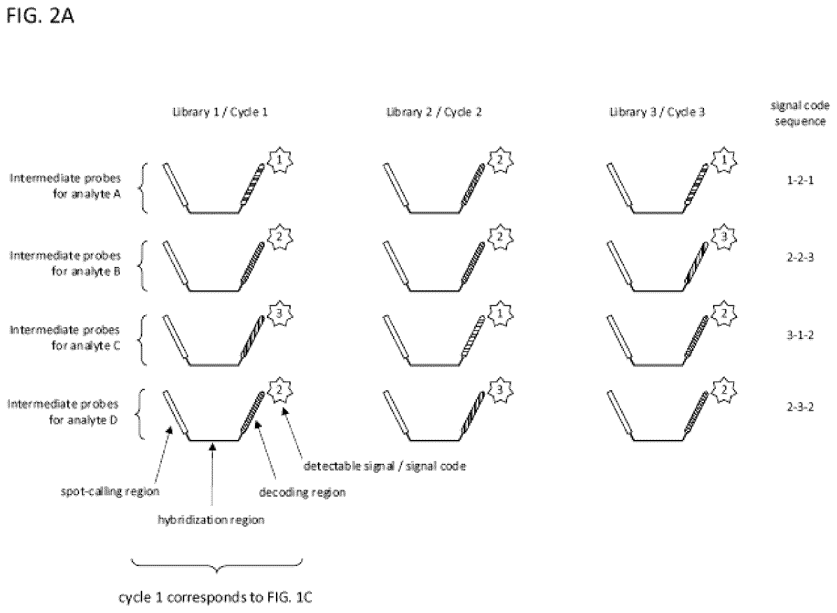

FIGS. 1A-1D show schematics illustrating exemplary intermediate probe libraries for spot-calling or spot-calling and decoding in a biological sample. Each panel shows an exemplary intermediate probe library. Each intermediate probe comprises (i) a hybridization region capable of hybridizing to the barcode region of a primary probe or a complement of the barcode region (not shown) and (ii) a spot-calling region. The barcode region of a primary probe is associated with the analyte to which the primary probe binds. Spot-calling regions of intermediate probes may be used to generate signals revealing the locations of analytes associated with the barcode regions or complements thereof hybridized by intermediate probes (e.g., spot-calling). Intermediate probe libraries in FIG. 1B and FIG. 1D comprise sub-libraries (e.g. a first sub-library and second sub-library) with corresponding spot-calling regions (e.g. first common spot-calling region and second common spot-calling region), which may be used for diluted spot-calling, as described herein. Intermediate probe libraries in FIG. 1C and FIG. 1D further comprise decoding regions. Decoding regions may be used for generating detectable signals associated with the barcode regions or complements thereof that are associated with specific analytes, for example at locations where spot-calling signals are detected. Different decoding regions can be used to generate different corresponding detectable signals that are used for decoding as described herein.

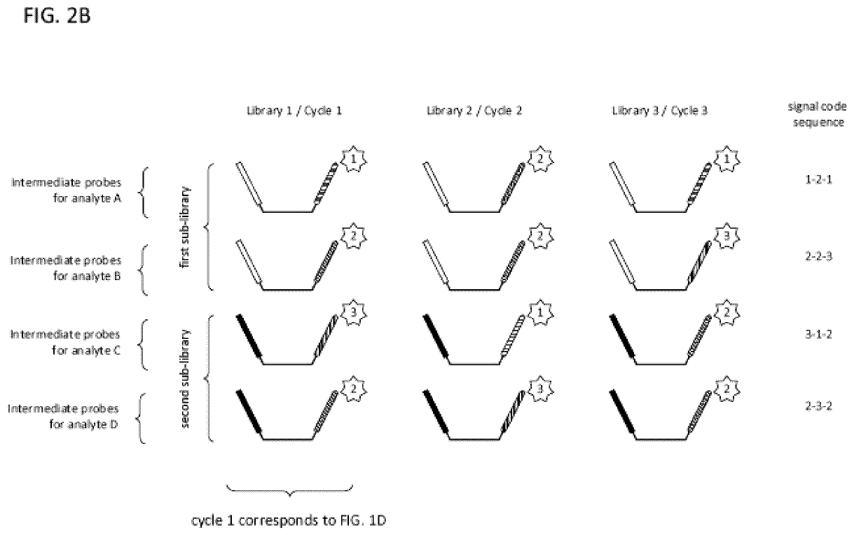

FIGS. 2A-2B show schematics illustrating exemplary intermediate probe libraries for sequential spot-calling and decoding cycles in a biological sample. Each column of probes is an intermediate probe library for one cycle. Probes in the same row comprise a hybridization region for hybridizing to a primary probe barcode region or product thereof associated with a particular analyte (e.g., analyte A in the top row). In an exemplary cycle, the intermediate probe library is hybridized to a plurality of primary probes or products thereof (e.g., rolling circle amplification (RCA) products of the primary probes). The spot-calling region is used to generate and detect a detectable signal (e.g., spot-calling signal) at multiple locations in the biological sample, thereby detecting the locations of the analytes associated with the barcode regions or complements thereof hybridized by the intermediate probes. The decoding regions are used to generate and detect detectable signals (decoding signals) at subsets of the multiple locations, the decoding signals being associated with the barcode regions or complements thereof associated with specific analytes. Different decoding regions may be used to generate different corresponding detectable signals, (e.g., detectable signals 1, 2, and 3 in FIG. 2A). Decoding signals that are detected in sequential cycles at a single location may delineate a signal code sequence corresponding to a particular analyte. For example, as shown in FIG. 2A, each of the intermediate probes in the top row may hybridize to the same primary probe barcode region or product thereof in sequential cycles, and generate a signal code sequence of 1-2-1, which corresponds to analyte A.

FIG. 2A shows a schematic illustrating exemplary intermediate probe libraries for sequential spot-calling and decoding cycles in a biological sample, wherein the intermediate probes from each library comprise the same spot-calling region and spot-calling is not diluted.

FIG. 2B shows a schematic illustrating exemplary intermediate probe libraries for sequential diluted spot-calling and decoding cycles in a biological sample. Each intermediate probe library comprises two sub-libraries, shown as a first sub-library and second sub-library. The intermediate probes in each sub-library comprise a corresponding spot-calling region. Different spot-calling regions can be used to generate different signals to facilitate diluted spot-calling within a cycle.

In some embodiments, the signals associated with the spot-calling region are associated with signals or absence thereof associated with the barcode regions detected. In some embodiments, the spot-calling signals are used for image registration (e.g., of images of decoding signals). In some embodiments, the spot-calling signals can used to overlay decoding signals that are detected. In some embodiments, the location of the signals detected in spot-calling are used to process and/or analyze signals detected from the decoding cycles.

In some aspects, analytes are targeted by primary probes, which are barcoded through the incorporation of one or more barcode sequences (e.g., sequences that can be detected or otherwise “read”) that are separate from a sequence in a primary probe that directly or indirectly binds the targeted analyte. In some aspects, the primary probes are in turn targeted by intermediate probes, which may also be barcoded through the incorporation of one or more barcode sequences that are separate from a recognition sequence in an intermediate probe that directly or indirectly binds a primary probe or a product thereof. In some embodiments, an intermediate probe may bind to a barcode sequence in the primary probe. In some embodiments, an intermediate probe comprises a region for binding to one or more detectably labeled probes (e.g., fluorescently labeled detection oligonucleotides which can be used as detectable probes for spot-calling or decoding as described herein). In some aspects, higher order probes may be used to target the intermediate probes, e.g., at a barcode sequence or complement thereof in an intermediate probe or product thereof. In some embodiments, the higher order probes may comprise one or more barcode sequences and/or one or more detectable labels. In some embodiments, for example, a tertiary probe is a detectably labeled probe that hybridizes to a barcode sequence (or complement thereof) of a secondary or intermediate probe (or product thereof). In some embodiments, through the detection of signals associated with detectably labeled probes in a sample, the location of one or more analytes in the sample and the identity of the analyte(s) can be determined. In some embodiments, the presence/absence, absolute or relative abundance, an amount, a level, a concentration, an activity, and/or a relation with another analyte of a particular analyte can be analyzed in situ in the sample.

C. Methods of Analyzing a Biological Sample Using Intermediate Probe Libraries

In some aspects, provided herein is a method for analyzing a biological sample to detect a plurality of analytes such as nucleic acids. In some embodiments, the method comprises performing spot-calling (e.g. the detection of signals corresponding to all or a subset of analytes of interest at locations in the sample) and decoding (e.g. the detection of signals (a signal signature) that together determine the identity of analytes in the locations). In some embodiments, the spot-calling is diluted spot-calling, for example wherein the locations of different subsets of analytes of interest are determined separately (e.g. at different times or in different fluorescent channels), thereby reducing optical crowding which can arise in methods using non-diluted spot-calling. In some embodiments, the spot-calling is performed using intermediate probes which provide advantages related to assay design and flexibility. In some embodiments, provided herein are nucleic acid probes that facilitate the methods comprising spot-calling and decoding.

In some aspects, provided herein is a method for analyzing a biological sample. In some embodiments, the method comprises contacting the biological sample with a plurality of primary probes. In some embodiments, each primary probe comprises (i) a binding region configured to bind to an analyte, and (ii) a barcode region associated with the analyte. In some embodiments, the method further comprises contacting the biological sample with a library of intermediate probes. In some embodiments, each intermediate probe comprises (i) a hybridization region configured to hybridize to the barcode region of a primary probe of the plurality of primary probes or a complement of the barcode region, (ii) a spot-calling region, and optionally (iii) a decoding region. In some embodiments, each intermediate probe comprises a decoding region. In some embodiments, each intermediate probe does not comprise a decoding region. In some embodiments, two or more of the intermediate probes in the library comprise a common spot-calling region. In some embodiments, the method further comprises detecting signals associated with the spot-calling regions of the library of intermediate probes at multiple locations in the biological sample, thereby detecting the locations of the analytes associated with the barcode regions or complements thereof hybridized by the library of intermediate probes. In some embodiments, the method comprises detecting signals or absence thereof associated with the barcode regions or complements thereof at at least a subset of the multiple locations.

In some embodiments, the hybridization region of the intermediate probe hybridizes to the barcode region of the primary probe. In some embodiments, the hybridization region of the intermediate probe hybridizes to a complement of the barcode region of the primary probe.

In some embodiments, the method comprises a step of generating a product of the primary probe. In some embodiments, the product comprises the barcode region of the primary probe or a complement thereof. For example, in some embodiments, the product is a rolling circle amplification (RCA) product. In some embodiments, the RCA product comprises multiple copies of the complement of the barcode region. For example, the primary probe can be a circularizable probe that is circularized and used as a template for RCA, thereby generating an RCA product comprising multiple copies of the complement of the primary probe barcode region. In other embodiments, the RCA product comprises multiple copies of the barcode region itself. For example, the barcode region of the primary probe can be hybridized by a secondary circularizable probe that is circularized and serves as a template for RCA, thereby generating an RCA product comprising multiple copies of the barcode region.

In some embodiments, the method can include one or more wash steps. Wash steps can be performed to remove unbound and/or nonspecifically bound probes (e.g. primary, intermediate, or detectable probes or products thereof) from the biological sample. Wash steps can include stringent wash to remove the unbound and/or nonspecifically bound probes. The one or more wash step can occur before, after, or during, any of the steps of the method provided herein. For example, a wash step can occur before or after hybridization of any of the probes provided herein, or before or after a detection/imaging step.

In some embodiments, the method comprises detecting signals (e.g. spot-calling signals) associated with detectable probes that are hybridized to the spot-calling regions of the library of intermediate probes. In some embodiments, the detectable probes hybridized to the spot-calling regions are fluorescently labeled and/or configured to directly or indirectly bind to fluorescently labeled probes. In some embodiments, the method comprises detecting signals (e.g. decoding signals) associated with detectable probes that are hybridized to the barcode regions or complements thereof, and/or detecting signals associated with detectable probes that are hybridized to decoding regions of intermediate probes, which are in turn hybridized to the barcode regions or complements thereof.

In some embodiments, the method comprises detecting signals associated with detectable probes, such as any of the detectable probes provided herein. For example, in some embodiments, the method comprises detecting signals associated with detectable probes hybridized to the spot-calling regions, detectable probes hybridized to the barcode regions or complements thereof, detectable probes hybridized to decoding regions of intermediate probes (e.g. any of the intermediate probes provided herein, including intermediate probes, decoding intermediate probes, subsequent decoding intermediate probes) which are in turn hybridized to the barcode regions or complements thereof. In some embodiments, any of the detectable probes provided herein can be fluorescently labeled, and/or configured to directly or indirectly bind to fluorescently labeled probes. The fluorescent label of the detectable probe or fluorescently labeled probe bound thereto can be used to generate a signal associated with the detectable probe, which in turn can be associated with an analyte (or plurality of analytes, e.g. in the case of a detectable probe hybridized to a spot-calling region).

In some embodiments, all of the intermediate probes in the library comprise the same common spot-calling region (e.g. as shown in FIG. 1A or FIG. 1C). In such embodiments, the intermediate probes can be used for spot-calling without diluted spot-calling (e.g. as shown in FIGS. 3A-3B).

In other embodiments, the intermediate probes in the library can comprise different spot-calling regions, which can correspond to different analytes. For example, in some embodiments, the library of intermediate probes comprises a first sub-library and second sub-library. In some embodiments, the intermediate probes in the first sub-library comprise a first common spot-calling region, the intermediate probes in the second sub-library comprise a second common spot-calling region, and the first and second common spot-calling regions are different. Similarly, the library of intermediate probes can comprise 3 sub-libraries, 4 sub-libraries, 5-sublibraries, or more sub-libraries, wherein each sub-library comprises its own common spot-calling region that is different from the spot-calling regions of the other sub-libraries. Sub-libraries (and associated spot-calling regions) can be associated with different sets of analytes. For example, in some embodiments, a first sub-library hybridizes to barcodes associated with a first set of analytes, and a second sub-library hybridizes to barcodes associated with a second set of analytes. Intermediate probe libraries with two or more sub-libraries having different common spot-calling regions can be used to perform diluted spot-calling. For example, spot-calling can be performed separately for each sub-library associated with a different set of analytes. The separate spot-calling, e.g. spot-calling using the first sub-library and second sub-library, can be performed sequentially (e.g. at different times) and/or in different channels (e.g. using different fluorophores associated with detectable probes hybridized to the first and second sub-library).

In some embodiments, when the library of intermediate probes comprises a first sub-library and second sub-library as described above, the method can comprise detecting signals associated with the first common spot-calling region of the first sub-library of intermediate probes at multiple locations in the biological sample, thereby detecting the locations of the analytes associated with the barcode regions or complements thereof hybridized by the first sub-library of intermediate probes. The method can also comprise detecting signals associated with the second common spot-calling region of the second sub-library of intermediate probes at multiple locations in the biological sample, thereby detecting the locations of the analytes associated with the barcode regions or complements thereof hybridized by the second sub-library of intermediate probes. In some embodiments, the method comprises contacting the biological sample with detectable probes that hybridize to the first common spot-calling region in the first sub-library of intermediate probes; and/or contacting the biological sample with detectable probes that hybridize to the second common spot-calling region in the second sub-library of intermediate probes. In some embodiments, the method comprises detecting signals associated with the barcode regions or complements thereof (e.g. decoding signals) at at least a subset of the locations detected using the first common spot-calling region of the first sub-library. In some embodiments, the method comprises detecting signals associated with the barcode regions or complements thereof (e.g. decoding signals) at at least a subset of the locations detected using the second common spot-calling region of the first sub-library. In some embodiments, the first sub-library comprises intermediate probes configured to hybridize to the barcode regions of primary probes or complements thereof associated with two or more different analytes. In some embodiments, the second sub-library comprises intermediate probes configured to hybridize to the barcode regions of primary probes or complements thereof associated with two or more different analytes. In some embodiments, one or more intermediate probes in the first sub-library and one or more intermediate probes in the second sub-library are configured to hybridize to the barcode regions of primary probes or complements thereof associated with different analytes.

In some embodiments, the method further comprises contacting the biological sample with a library of decoding intermediate probes, wherein each decoding intermediate probe comprises (i) a hybridization region configured to hybridize to the barcode region of a primary probe of the plurality of primary probes or a complement of the barcode region, and (ii) a decoding region. The decoding intermediate probes do not need to include a spot-calling region. For example, spot-calling can be performed prior to and/or after contacting the biological sample with the library of decoding intermediate probes. In some embodiments, the library of decoding intermediate probes can be used to generate decoding signals that contribute to the generation of a signal code sequence used to identify analytes, as described herein. In some embodiments, the method comprises contacting the biological sample with detectable probes that hybridize to the decoding regions of the library of decoding intermediate probes, and detecting signals or absence thereof associated with the detectable probes hybridized to the decoding regions of the library of decoding intermediate probes. In some embodiments, the method comprises contacting the biological sample with a subsequent library of decoding intermediate probes, wherein each subsequent decoding intermediate probe comprises (i) a hybridization region configured to hybridize to the barcode region of a primary probe of the plurality of primary probes or a complement of the barcode region, and (ii) a subsequent decoding region. Like the decoding intermediate probes, the subsequent library of decoding intermediate probes does not need to include a spot-calling region. In some embodiments, the method comprises contacting the biological sample with subsequent detectable probes that hybridize to the subsequent decoding regions of the subsequent library of decoding intermediate probes, and detecting signals or absence thereof associated with the subsequent detectable probes hybridized to the subsequent decoding regions. In some embodiments, the decoding intermediate probes and the subsequent decoding intermediate probes hybridize to the same barcode sequence (or complement thereof) or different barcode sequences (or complements thereof) of the barcode region, optionally wherein the different barcode sequences are partially overlapping. In some embodiments, the decoding region and the subsequent decoding region are configured to hybridize to the same detectable probe. In some embodiments, the decoding region and the subsequent decoding region are identical in sequence and hybridize to detectable probes comprising the same detectable label. In some embodiments, the decoding region and the subsequent decoding region are configured to hybridize to detectable probes of different sequences. In some embodiments, each detectable probe of a different sequence comprises a different detectable label.

In some embodiments, the intermediate probes in the library of intermediate probes comprise decoding regions. In some aspects, the decoding regions can be used to generate signals (e.g. decoding signals) that are used to determine the identity of analytes, for example by generating a signal code sequence at a particular location, as described herein. The decoding regions of the library of intermediate probes can be used to generate decoding signals in one sequential round of hybridization and detection, and subsequent rounds of hybridization and detection can utilize another library of intermediate probes (such as intermediate probes with or without a spot-calling region) to generate additional decoding signals, which ultimately are used to generate the signal code sequences.

In some embodiments, the method comprises contacting the biological sample with detectable probes that hybridize to the decoding regions of the library of intermediate probes and detecting signals or absence thereof associated with the detectable probes hybridized to the decoding regions of the library of intermediate probes. In some embodiments, the detectable probes hybridized to the decoding regions of the library of intermediate probes are fluorescently labeled and/or configured to directly or indirectly bind to fluorescently labeled probes.

In some embodiments, the method comprises generating a signal code sequence for one or more of the multiple locations. The signal code sequence can comprise signal codes corresponding to: the signals (or absence thereof) associated with the detectable probes hybridized to the decoding regions of the library of intermediate probes, the signals (or absence thereof) associated with the detectable probes hybridized to the decoding regions of the library of decoding intermediate probes, and/or the signals (or absence thereof) associated with the subsequent detectable probes hybridized to the decoding regions of the library of subsequent decoding intermediate probes. In some embodiments, the signal code sequence corresponds to an analyte. In some embodiments, generation of the signal code sequence allows detecting the analyte at the one or more of the multiple locations.

In some embodiments, the barcode region comprises one or more barcode sequence or a combination of barcode sequences. In some embodiments, the barcode sequence or the combination of barcode sequences corresponds to the analyte associated with the barcode region.

In some embodiments, each primary probe can have any suitable configuration of components of a primary probe described herein. In some embodiments, each primary probe is independently selected from the group consisting of: a primary probe comprising a 3′ or 5′ overhang, optionally wherein the 3′ or 5′ overhang comprises one or more barcode sequences; a primary probe comprising a 3′ overhang and a 5′ overhang, optionally wherein the 3′ overhang and the 5′ overhang each independently comprises one or more barcode sequences; a circular primary probe; and a circularizable primary probe or probe set.

In some embodiments, the each intermediate probe can have any suitable configuration of components of an intermediate probe described herein. In some embodiments, each intermediate probe is independently selected from the group consisting of: an intermediate primary probe comprising a 3′ or 5′ overhang, optionally wherein the 3′ or 5′ overhang comprises one or more spot-calling regions and/or decoding regions; an intermediate probe comprising a 3′ overhang and a 5′ overhang, optionally wherein the 3′ overhang and the 5′ overhang each independently comprise one or more spot-calling regions and/or decoding regions; a circular intermediate probe; and a circularizable intermediate probe or probe set.

### IV. Signal Detection and Decoding

In some embodiments, a method disclosed herein comprises performing multiple cycles of decoding using a plurality of probes, such as a library of intermediate probes as described herein (e.g., using the probes in a pre-determined order), wherein each cycle comprises contacting the analytes (e.g., a signal amplification product such as rolling circle amplification products) with the probes, allowing the probes to bind to their respective analytes, and detecting a signal from the probes which have bound to the analytes (e.g. a decoding signal and/or a spot-calling signal).

In some embodiments, a spot-calling signal and/or a decoding signal is generated from an intermediate probe bound to an analyte (or a detectable probe associated with the intermediate probe), simultaneously or sequentially. In some embodiments, spot-calling signals are used to map the locations of all or a subset of analytes. In some embodiments, decoding signals are used to generate fluorescent readouts in sequential cycles that delineate a signal code sequence, thereby revealing the identity of a specific analyte in a specific location in the biological sample, as described below.

In some embodiments, the same spot-calling signal is generated from each of the intermediate probes associated with different analytes (e.g. detecting a common spot-calling region using a single fluorophore). In some embodiments, different spot-calling signals are generated for different subsets of the intermediate probes associated with different analytes (e.g. detecting different spot-calling regions using different fluorophores).

In some embodiments, in one or more cycles, the probes for one or more analytes (e.g., selected target nucleic acid sequences), and/or the detectable labels thereof, are not detected, such that spot-calling signals from the one or more analytes are not detected in those cycles, thereby reducing signal crowding. In some embodiments, the hybridized probes and/or the detectable labels thereof are removed between cycles.

In some embodiments, provided herein is a method for analyzing a biological sample comprising a plurality of analytes, the method comprising detecting a plurality of optical signals in sequential cycles, wherein at least a subset of the plurality of optical signals detected at a location in the biological sample form an optical signature corresponding to an analyte of the plurality of analytes. In some embodiments, provided herein is a method for analyzing a biological sample comprising a plurality of analytes, the method comprising detecting optical signals in one or more imaging rounds for spot-calling, wherein optical signals are detected at locations in the biological sample and can be used for image registration, and the locations of signals detected in the spot-calling round can be used in the analysis of decoding signals (e.g., optical signals from sequential cycles). In some embodiments, spot-calling signals associated with a first plurality of analytes are detected in a round of spot-calling, and spot-calling signals are “dark” for a second plurality of analytes—that is, signals associated with the second plurality of analytes are not detected in that round. In some embodiments, spot-calling signals for different pluralities of analytes are detected in different channels (e.g. fluorescent colors) and/or detected at different times.

In some embodiments, a plurality of analytes can be decoded (e.g., identified from other analytes) by contacting a sample containing or suspected of containing the analyte with detectably labeled probes that recognize the barcode regions of primary probes or products thereof to produce probe binding patterns in sequential probe binding cycles, whereby patterns of detectably labeled probe binding can be compared to a list of known and/or allowed order of signal codes (e.g., identifiers) corresponding to the analytes (e.g., a codebook or “whitelist”). In some embodiments, the list of known order of signal codes (e.g., identifiers) corresponding to the analytes includes one or more dark cycles.

In some embodiments, decoding comprises compiling, processing, and analyzing a plurality of optical signals detected from sequential cycles at a location in a biological sample. In some aspects, the optical signals detected from one or more of the sequential cycles may include both spot-calling and decoding signals (e.g. signals generated and detected using spot-calling regions or a decoding regions, respectively, of intermediate probes). In some instances, if a cycle comprises both a spot-calling step and a decoding step, one or more colors are reserved for spot-calling (e.g., the reserved color(s) is not used for decoding signals). In some aspects, a location comprising optical signals comprises optical signals from at least two neighboring spots (e.g., two analytes, such as two RCPs) that are close in proximity (e.g., optically overlapping). In some embodiments, a location may comprise two or more neighboring spots that are within 1 μm distance. In some embodiments, a location may comprise two or more neighboring spots that are within about 0.5 to 1 μm distance. In some cases, each location including all spots (e.g., overlapping spots) within that distance is observed and potential signal sequence chains are generated for this location. In some aspects, the optical signals from neighboring spots are detected and processed to determine which optical signals are associated with the first of the neighboring spots vs. the optical signals that are associated with the second of the neighboring spots. In some cases, an optical signal can be a presence of a signal (e.g., a detected signal in a channel) or an absence of a signal in the location. In some cases, the processing includes compiling the detected optical signals. In some cases, the processing includes comparing observed optical signals from various sequential cycles of detection to a list of known order of signal codes (e.g., of the identifiers) corresponding to the analytes (e.g., from a codebook or “whitelist”). In some cases, optical signals corresponding to the analyte are processed including spot-calling and/or decoding signals.

In an exemplary workflow, RCA products (RCPs) at a location in a biological sample can be identified from individual channels in different cycles. In some embodiments, for each RCP, its nearest neighbors within 1 μm distance across all images can be identified, and only the closest neighbor from each image cycle is kept. In some examples, a decoding signal corresponding to an RCP in the sample is aligned with or assigned a location determined from a spot-calling signal. In some examples, decoding signals detected in sequential cycles corresponding to the same RCP are aligned with or assigned a location determined based on one or more spot-calling signals.

In some embodiments, provided herein is a method for nucleic acid sequence detection, comprising: (a) contacting a sample with multiple sets of probes (e.g., intermediate probes) in sequential cycles, wherein each set is specific for a corresponding target nucleic acid sequence (e.g. barcode or complement thereof) and each set comprises multiple probes that (i) hybridize to a particular nucleic acid sequence and (ii) are directly or indirectly labeled with a detectable label which may be the same or different for probes in the same probe set, wherein the sequential cycles comprise contacting the sample with probes within a set in a pre-determined order which corresponds to a signal code sequence for the corresponding target nucleic acid sequence; and (b) obtaining a signal code sequence for each target nucleic acid sequence, wherein the signal code sequence comprises signal codes corresponding to the signals or absence thereof from detectable labels for probes in the sequential cycles, thereby detecting the target nucleic acid sequences in the sample.

In some embodiments, described herein is a method of localized detection of multiple target nucleic acid sequences in a sample, wherein each target nucleic acid sequence is detected using a circularizable primary probe specific for said target sequence which is circularized upon hybridization to the target sequence and is amplified by rolling circle amplification (RCA) to produce a rolling circle product (RCP), each circularizable primary probe comprising a different barcode region specific for a different target nucleic acid sequence, and each RCP containing multiple complementary copies of the barcode region, wherein the barcode region is decoded in multiple sequential decoding cycles each using hybridization probes (e.g. intermediate probes) which hybridize to the complementary copies of the barcode region in an RCP and allow detectable signals to be generated which together yield a unique signal code sequence which identifies the target nucleic acid sequence.

In some embodiments, provided herein is a method of analyzing a sample, comprising: a) producing a product (e.g., an amplification product such as RCA product; a branched DNA product, an HCR product, or a PER product, etc.) in the sample, the product (e.g., amplification product) comprising multiple copies of a barcode sequence or complement thereof, wherein the barcode sequence is associated with a target analyte and is assigned a signal code sequence, and wherein the sample is a cell or tissue sample; b) contacting the sample with a first intermediate probe and a first detectable probe to generate a first complex comprising the first intermediate probe hybridized to the product (e.g., amplification product) and the first detectable probe hybridized to the first intermediate probe, wherein the first intermediate probe comprises (i) a hybridization region complementary to the barcode sequence and (ii) a first overhang sequence, and wherein the first detectable probe comprises (i) a sequence complementary to the first overhang sequence and (ii) a first optically detectable moiety; c) imaging the sample to detect a first signal from the first optically detectable moiety, wherein the first signal corresponds to a first signal code in the signal code sequence; d) contacting the sample with a second intermediate probe and a second detectable probe to generate a second complex comprising the second intermediate probe hybridized to the product (e.g., amplification product) and the second detectable probe hybridized to the second intermediate probe, wherein the second intermediate probe comprises (i) a hybridization region complementary to the barcode sequence and (ii) a second overhang sequence, and wherein the second detectable probe comprises (i) a sequence complementary to the second overhang sequence and (ii) a second optically detectable moiety; and e) imaging the sample to detect a second signal from the second optically detectable moiety, wherein the second signal corresponds to a second signal code in the signal code sequence, wherein the signal code sequence comprising at least the first signal code and the second signal code is determined at a location in the sample, thereby decoding the barcode sequence and identifying the target analyte at the location in the sample. In some embodiments, the barcode sequence associated with the target analyte is selected from a plurality of barcode sequences, wherein the method comprises contacting the sample with a first pool of intermediate probes and a universal pool of detectable probes, wherein the first pool of intermediate probes comprises the first intermediate probe and the universal pool of detectable probes comprises the first detectable probe and the second detectable probe, wherein each intermediate probe in the first pool of intermediate probes comprises (i) a hybridization region complementary to one of the plurality of barcode sequences and (ii) an overhang sequence complementary to a detectable probe of the universal pool of detectable probes; and the method comprises contacting the sample with a second pool of intermediate probes and the universal pool of detectable probes, wherein the second pool of intermediate probes comprises the second intermediate probe, and wherein each intermediate probe in the second pool of intermediate probes comprises (i) a hybridization region complementary to one of the plurality of barcode sequences and (ii) an overhang sequence complementary to a detectable probe of the universal pool of detectable probes.

In some embodiments, the method comprises identifying multiple different target analytes present at locations in the sample, wherein each different target analyte is assigned a different signal code sequence (e.g., signal signature) and is targeted by a circularizable probe or probe set comprising a complement of a different barcode sequence of the plurality of barcode sequences. In some embodiments, the number of different intermediate probes in each pool of intermediate probes is greater than the number of different detectable probes in the universal pool of detectable probes. In some embodiments, the number of different detectable probes in the universal pool of reporter probes is four. In some embodiments, the number of different intermediate probes in each pool of intermediate probes is about 10, about 20, about 50, about 100, about 200, about 500, about 1,000, about 2,000, about 5,000, or more. In any of the embodiments herein, the pool of intermediate probes can comprise one or more intermediate probes each comprising a spot-calling region (e.g., in an overhang sequence of the intermediate probe), and the universal pool of detectable probes can comprise detectable probes that hybridize to the spot-calling regions. Signals associated with the spot-calling regions can be used to facilitate registration of signals detected in the sequential cycles for decoding. Moreover, the intermediate probes may comprise different subsets of spot-calling regions that can be detected separately, thereby alleviating signal crowding (e.g., overlapping of signals for spot-calling).

In some embodiments, a sequential decoding scheme involves detecting sequential signals from a given target (e.g. analyte) in multiple cycles, and the target may be in the same position in the sample in the different cycles. In some embodiments, a method disclosed herein comprises the localized detection of analytes (e.g. target nucleic acid sequences). In some embodiments, the analyte is present at a fixed or defined location in the sample, and is detected at that location. The analyte may be localized by virtue of being present in situ at its native location in the sample (e.g. a cell or tissue sample), or of being attached or otherwise localized to a target analyte which is present in situ at its native location in the sample. The analyte may be immobilized in the sample. Thus, a target analyte and/or a target nucleic acid sequence may be artificially immobilized in the sample, e.g. fixed on or to a solid surface, or bound to an immobilizing moiety provided on a solid surface.

In some embodiments, the number of decoding cycles that are required to fully identify all of the individual signal codes which make up the signal code sequence for all of the analytes is greater than the number of individual signal codes which make up the signal code sequences. In some embodiments, the number of decoding cycles required to fully identify all of the signal code sequences is greater than the number of hybridization probes in at least one set of hybridization probes, or greater than the number of signal code positions in a least one signal code sequence. Generally speaking the signal code sequences for different targets are designed to be of the same length, but this is not essential.

In some embodiments, provided herein is a method comprising detecting analytes such as target nucleic acid sequences by primary probes, or circularizable probes or probe sets more generally, wherein the primary probe is detected by detecting a rolling circle amplification (RCA) product of the primary probe.

In some embodiments, each target nucleic acid sequence in the sample is detected using a circularizable probe specific for that target sequence which is circularized upon hybridization to the target sequence and is amplified by rolling circle amplification (RCA) to produce a rolling circle product (RCP). In some embodiments, each circularizable probe comprises a different barcode region specific for a different target nucleic acid sequence, and therefore each RCP contains multiple complementary copies of the barcode region.

In some embodiments, the barcode region is decoded in multiple sequential decoding cycles. In some embodiments, the barcode region corresponds to a unique signal code sequence which is specific to the target nucleic acid sequence. In some embodiments, each decoding cycle uses hybridization probes (e.g. intermediate probes) which hybridize to the complementary copies of the barcode region in the RCP and comprise regions for generating detectable signals (e.g. decoding regions), wherein each signal corresponds to an individual signal code, and the signal codes together yield the unique signal code sequence (e.g., signal signature) which identifies the target nucleic acid sequence.

In some embodiments, image registration is performed. In some aspects, image registration comprises aligning signals and/or images obtained from various cycles onto a common coordinate system. When obtaining images or detecting signals from a sample across multiple cycles, the sample or imaging apparatus may shift, causing an offset of images from one cycle to the next. In some aspects, image registration compensates for these shifts, allowing the user to identify the same relative location within the sample between different images, and/or overlay images that are spatially aligned. In some embodiments, spot-calling signals are used for image registration. In some embodiments, spot-calling signals provide a plurality of physical landmarks within the sample that can be used to align multiple images. In some embodiments, decoding signals can be assigned to locations associated with spot-calling signals. In some embodiments, image registration allows decoding signals from multiple cycles to be assigned to the same location (e.g. a location associated with a spot-calling signal), allowing a signal code sequence to be constructed for that location. In some embodiments, image registration is performed using computational methods. In some embodiments, image registration comprises various image processing steps. In some cases, the processing comprises extraction and/or identification of features in the image of the sample. In some cases, the extraction comprises uses an automatic feature extraction algorithm. In some cases, feature extraction results in keypoints (e.g., landmarks) in two or more images of the sample (e.g., an image of spot calling signals and an image of decoding signals). In some aspects, features that are common across two or more images are used to align the images. In some embodiments, image registration is performed manually, is guided, or is adjusted by a user.

In some embodiments, sequential hybridization and imaging cycles are performed using an instrument to identify the locations of target nucleic acid analytes in the sample, and signals are detected and analyzed to reveal the identities of target nucleic acid analytes at the locations. Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., sequential hybridization of detectable probes). Reagents for sequential cycles comprising spot-calling, decoding, or both spot-calling and decoding, can be delivered in any order. Additionally, the optics module can be configured to illuminate the biological sample with light having one or more spectral emission curves (over a range of wavelengths) and subsequently capture one or more images of emitted light signals from the biological sample during one or more probing cycles (e.g., as described in Section III and IV). In various embodiments, the captured images may be processed in real time and/or at a later time to determine the presence of the one or more target molecules in the biological sample, as well as three-dimensional position information associated with each detected target molecule. Additionally, the opto-fluidics instrument can include a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to detect and/or analyze one or more target molecules (e.g., as described in Section II) in their naturally occurring position (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample (e.g., spot-calling signal and decoding signals). The in-situ analysis system may also include other ancillary modules configured to facilitate the operation of the opto-fluidic instrument, such as, but not limited to, cooling systems, motion calibration systems, etc.

In various embodiments, the opto-fluidic instrument may analyze the sample and may generate the output that includes indications of the presence of the target molecules in the sample. For instance, with respect to the example embodiment discussed above where the opto-fluidic instrument employs a hybridization technique for detecting molecules, the opto-fluidic instrument may cause the sample to undergo decoding cycles of hybridizing detectable probes and detecting signals to generate a signal signature (corresponding to an analyte in the sample). In such cases, the output may include optical signal signatures (e.g., a codeword) specific to each analyte (e.g., associated with a primary probe described herein), which allow the identification of the target nucleic acids.

### V. Signal Crowding

In some embodiments, the present disclosure addresses signal crowding in methods that involve detecting signals, e.g., from nucleic acid sequences (either as the target analytes or as the labels or reporters for one or more target analytes, such as one or more target proteins), including in situ assays that detect the localization of analytes in sample. There are a number of situations in which it is desired to detect several different analytes in a sample simultaneously, for example when detecting the expression of different genes in situ in a sample. In some embodiments, nucleic acid molecules are detected as target analytes in situ in a sample. In some embodiments, nucleic acid molecules are detected as reporters for other, non-nucleic acid analytes, including for example proteins, or indeed as a reporter, or signal amplifier, for a nucleic acid analyte.

In some embodiments, a method disclosed herein comprises generating and detecting localized signals associated with analytes in a biological sample in order to identify the locations and identities of the analytes. In some embodiments, a spot-calling signal is generated which corresponds to all or multiple different analytes in the sample. In some embodiments, spot-calling signals are used to map the locations of the analytes. In some embodiments, spot-calling signals are used for image registration. In some aspects, if a large number of analytes, or a high concentration of one or more analytes is present in the sample, then a high concentration of spot-calling signals will be generated. Consequently, spot-calling signals in close proximity may be overcrowded and/or overlapping with one another, making it difficult or impossible to distinguish the locations of individual signals. In some embodiments, the methods disclosed herein can prevent and/or ameliorate crowding of spot-calling signals in multiplex assays where it is desired to detect a number of different analytes that are in close proximity and/or present in high concentrations.

In some embodiments, a method disclosed herein comprises detecting and identifying RNA transcripts in a given cell, in order to analyze the gene expression of that cell. In some embodiments, a method disclosed herein comprises labeling the RNA transcripts (or one or more primary or higher order probes bound thereto) with fluorescently labeled probes. The signals from the fluorescent labels can then be visualized in order to determine which RNA transcripts are present in a given cell of, e.g., a tissue sample. This can also be used to provide information on the location and the relative quantities of different RNA transcripts (and therefore the location and relative levels of expression of the corresponding genes). If a particular gene (or genes) is significantly overexpressed, or a large number of genes are analyzed, then a large number of RNA transcripts corresponding to those genes will be present in the sample, and thus a large number of fluorescent signals indicating the presence of the RNA transcripts will be generated, including spot-calling signals. At a certain point, the signal density will be such that at least some individual signals cannot be resolved using conventional fluorescence microscopy, which leads to a loss of information and an inaccurate picture of gene expression. It will be understood that this problem can occur in many other nucleic acid detection methods. In some aspects, the present disclosure provides methods of mapping the locations of multiple analytes (e.g., spot-calling) in a sample wherein signal crowding is reduced.

In some embodiments, the methods provided in this disclosure are for use in the multiplexed detection of analytes (such as nucleic acids), that is, for the detection of multiple target analytes in a sample, e.g., one or more tissue samples such as a single tissue section or a series of tissue sections. In some embodiments, the methods use hybridization probes (e.g. intermediate probes), while reducing signal crowding from said hybridization probes. In some embodiments, the methods provided herein comprise sequencing-by-hybridization (SBH) for detecting nucleic acid sequences in a sample, including multiplex SBH for detecting different target nucleic acid sequences (e.g., labels or reporters for one or more target analytes), with a wide range of distribution and abundance simultaneously in a sample. In some embodiments, the methods provided herein comprise multiple rounds of probe hybridization and detection in order to identify the locations and identities of nucleic acid sequences in a sample. In some embodiments, one or more rounds of probe hybridization and detection include detection of a spot-calling signal, which is used to map the locations of all or a subset of the analytes of interest in the sample. In some embodiments, the methods provided herein address signal crowding issues due to signals indicative of target analytes present in high concentrations and/or close proximity that may mask and/or overcrowd one another and/or other signals. In some embodiments, the methods provided herein address signal crowding of spot-calling signals.

In some aspects, signal overcrowding may prevent signals relating to the target nucleic acid sequences from being generated, detected, analyzed and/or otherwise distinguished from one another and/or other signals in the sample. Signals (e.g. spot-calling signals) may be present at such high densities, either in a particular area of the sample or in the sample as a whole (e.g., the signal density is too large), that not all of the signals can be properly detected and resolved. Where the signals are detected by optical means, this may be referred to as optical crowding, and the present methods are particularly suited to resolving, or reducing, optical crowding. In some aspects, by “optical means” is meant that the signals are detected visually, or by visual means, namely that the signals are visualized. Thus, in some instances, the signals that are generated involve detection of light or other visually detectable electromagnetic radiation (such as fluorescence). In some aspects, the signals may be optical signals, visual signals, or visually detectable signals. The signals may be detected by sight, typically after magnification, but more typically they are detected and analyzed in an automated system for the detection of the signals.

In some aspects, the signals may be detected by microscopy. In some aspects, an image may be generated in which the signals may be seen and detected, for example an image of the field of view of a microscope, or an image obtained from a camera. The signals may be detected by imaging, more particularly by imaging the sample or a part or reaction mixture thereof. By way of example, signals in an image may be detected as “spots” which can be seen in the image. In some aspects, a signal may be seen as a spot in an image. In some aspects, optical crowding occurs when individual spots cannot be resolved, or distinguished from one another, for example when they overlap, or mask one another. By reducing the number of spots that are detected simultaneously (e.g. signals generated for spot-calling) using the methods herein, individual spots, or signals, can be resolved, and optical crowding can be reduced. In some aspects, the present methods optically de-crowd the signals.

In some embodiments, analyzing a biological sample comprises imaging the sample and analyzing a captured image. In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location (e.g. using decoding signals). For instance, a particular location in a sample can be tracked and decoding signals from sequential sequencing-by-hybridization cycles can be analyzed to detect a target polynucleotide sequence (e.g., a barcode region, barcode sequence or subsequence thereof) in a nucleic acid at the location. The decoding signals may be aligned to a spot-calling signal detected in one or more of the cycles. The analysis may comprise processing information of one or more cell types, one or more types of analytes, a number or level of analyte, and/or a number or level of cells detected in a particular region of the sample. In some embodiments, the analysis comprises detecting a sequence (e.g., a barcode region or complement thereof) present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some cases, the analysis further includes displaying the information from the analysis or detection step. In some embodiments, software may be used to automate the processing, analysis, and/or display of data.

In some aspects, the reduction in signal crowding associated with the present methods may be considered to be a reduction relative to the level of signal crowding which would occur in a method which did not comprise steps to reduce signal crowding, for example, without the use of intermediate probes with different spot-calling regions (e.g., diluted spot-calling) as described herein.

In some aspects, the methods herein involve reducing the number of signals (e.g. spot-calling signals) that are detected in a detection step of the method. In some aspects, the methods herein involve reducing the number of spot-calling signals that are detected using a specific detectable label (e.g. fluorophore) in a detection step. In some aspects, the methods herein involve performing diluted spot-calling, e.g., generating different spot-calling signals associated with different subsets of analytes, thereby generating the same total number of spot-calling signals while reducing the density of spot-calling signals from individual modalities.

In some embodiments, in situ analysis provided herein comprises multiple decoding cycles of hybridizing detectable probes and detecting signals to generate a signal signature (corresponding to an analyte in the sample). In some embodiments, in the decoding cycles, a subset of all analytes is imaged in each cycle to prevent optical crowding. In some embodiments, signals from the various images from different decoding cycles are aligned to generate the signal signature. In some cases, alignment of signals detected in various cycles and registration requires a stable optical stage such that there are no detectable shifts between the images from each of the cycles, which can be challenging. In some cases, alignment of signals detected in various cycles and registration can be performed using a computational approach using partial overlap of the detected signals from various decoding cycles. In some instances, the computational approach using partial overlap may be challenging to optimize because the level of partial overlap determines the quality of registration (e.g., too little overlap may be sub-optimal for registration and/or other registration errors may occur). Provided herein are methods that may provide certain benefits using universal or common spot-calling regions for registration such that the images obtained in the various decoding cycles can be mapped using the spot-calling signal(s) to align the signals detected in various decoding cycles to generate the signal signature. In some instances, the spot-calling signals are used to map the location of transcripts and used as a guide to register various decoding images.

FIGS. 3A-3D show schematics illustrating an exemplary cycle of spot-calling and decoding. As shown in FIG. 3A and FIG. 3C, each primary probe is hybridized to a different analyte and circularized, and comprises a barcode region associated with the analyte to which it binds. The circularized primary probes are amplified by RCA to generate RCA products comprising multiple copies of complements of the barcode regions. Intermediate probes corresponding to specific analytes are hybridized to the complement of the barcode region corresponding to the analyte. Each intermediate probe comprises a spot-calling region and a decoding region. All intermediate probes may comprise the same spot-calling region, for example as shown in FIG. 3A. Alternatively, intermediate probes may comprise sub-libraries, wherein each sub-library comprises a different common spot-calling region, for example as shown in FIG. 3C. Detectable probes for spot-calling are hybridized to the spot-calling regions of each intermediate probe, and detectable probes for decoding are hybridized to corresponding decoding regions. The detectable probes may be fluorescently labeled and/or configured to directly or indirectly bind to fluorescently labeled probes. In some embodiments, a detectable probe for spot-calling and a detectable probe for decoding in the same imaging cycle are different in sequences, e.g., one hybridizing to a spot-calling region and the other hybridizing to a decoding region different from the spot-calling region. A detectable probe for spot-calling may be the same in nucleic acid sequence as a detectable probe for decoding, as long as the detectable probe for spot-calling and the detectable probe for decoding used in the same imaging cycle are orthogonal. For example, the signals from the spot-calling detectable probe and the decoding detectable probe are different and do not crosstalk (e.g., the detectable probe probes can be labeled with different fluorophores). FIG. 3B and FIG. 3D show schematics illustrating exemplary imaged biological samples with locations of detected spot-calling and decoding signals generated using the intermediate probe library illustrated in FIG. 3A and FIG. 3C, respectively. The spot-calling signals may be undiluted (e.g. generated using a single common spot-calling region detected using a single fluorophore, as shown in FIG. 3A and FIG. 3B), or diluted (e.g. generated using different spot-calling regions detected using different fluorophores, as shown in FIG. 3C and FIG. 3D). Signal codes (e.g., shown as 1, 2, or 3 in the figure) are assigned to each decoding signal, and decoding signals may be aligned with subsets of spot-calling signals.

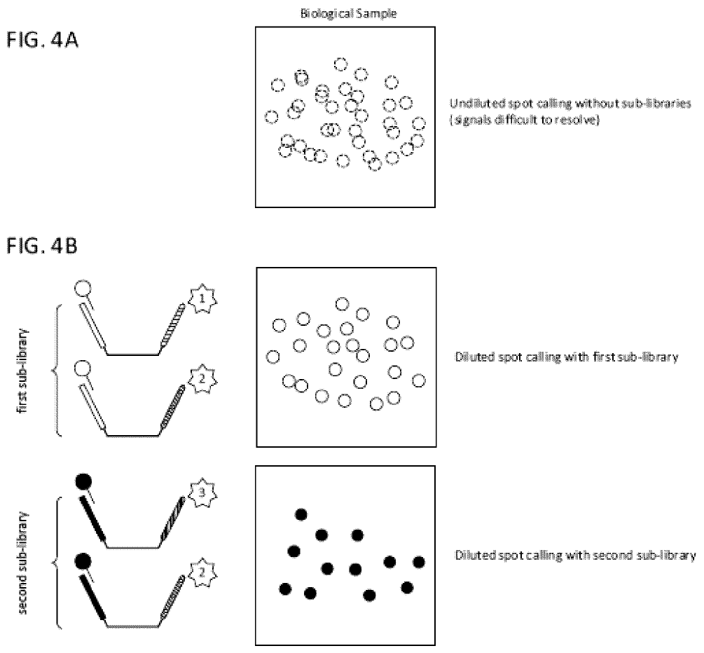

FIGS. 4A-4B show schematics illustrating examples of undiluted and diluted spot-calling in a biological sample with a high density of analytes that are crowded and/or overlapping. FIG. 4A shows the locations of signals detected using undiluted spot-calling (circles with dotted outlines). The signals are overlapping and difficult to resolve. FIG. 4B shows the locations of signals detected using diluted spot-calling with an exemplary intermediate probe library comprising sub-libraries with different common spot-calling regions. The spot-calling signals detected for each of the sub-libraries are different (e.g. detected using different fluorophores), resulting in diluted signals that are easily resolved (white and black circles).

In some embodiments, intermediate probes for different analytes that are known to be highly expressed in a biological sample (e.g., that may spatially overlap) are designed such that corresponding intermediate probes for these analytes are provided in different sub-libraries with different common spot-calling regions. In some embodiments, certain analytes may be known to be highly expressed in a biological sample (e.g., they may spatially overlap). Intermediate probes may be designed such that different sub-libraries of intermediate probes with different spot-calling regions correspond to different highly expressed analytes. For example, two highly expressed analytes can be analyzed by intermediate probes that belong to different sub-libraries, such that spot-calling signals associated with the two highly expressed analytes are not detected at the same time, thereby alleviating spatial overlapping of spot-calling signals and facilitating registration and detection of signals for decoding.

### VI. Signal Amplification, Detection and Analysis

In some embodiments, the present disclosure relates to the detection of analytes (e.g., nucleic acids sequences) in situ using probe hybridization. In some embodiments, the detection of analytes comprises generation of amplified signals associated with the probes (e.g., primary probes).

In some embodiments, detection comprises an amplification product of one or more polynucleotides, for instance, a circular probe or circularizable probe or probe set hybridized to an analyte. In some embodiments, the amplifying is achieved by performing extension or amplification such as rolling circle amplification (RCA), for example as described in Section II.B. and Section VI.

In some aspects, the provided methods involve analyzing, e.g., detecting or determining, one or more sequences present in a target nucleic acid, or probes directly or indirectly hybridized thereto. In some aspects, the provided methods involve analyzing one or more detectable signals associated with the target nucleic acid. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments, the detection may be spatial, e.g., in two or three dimensions. In some embodiments, the detection may be quantitative, e.g., the amount or concentration of detectable signals (and of a corresponding target nucleic acid) may be determined. In some embodiments, the primary probes, intermediate probes, higher order probes, and/or detectably labeled probes may comprise any of a variety of entities able to hybridize a nucleic acid, e.g., DNA, RNA, LNA, and/or PNA, etc., depending on the application.

Detectable probes may comprise recognition sequences that hybridize to sequences in the probes forming a hybridization complex with the target nucleic acid (e.g. primary probes hybridized to analytes, RCA products thereof, and intermediate probes), for example to barcode sequences or complements thereof, or decoding regions. The recognition sequences may be of any length, and multiple recognition sequences in the same or different probes of the hybridization complex may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof of a circular or circularized probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, a hybridized primary probe as described herein can be detected with a method that comprises signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is incorporated herein by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is incorporated herein by reference in its entirety), e.g., primer exchange reactions such as signal amplification by exchange reaction (SABER) or SABER with DNA-Exchange (Exchange-SABER). In some embodiments, a non-enzymatic signal amplification method may be used. In some embodiments, the primary probe is detected using RCA. In some cases, intermediate probes as described in Section III.B. and III.C. can be used to detect a sequence of a component of a signal amplification system.

In some embodiments, detection of a hybridized primary probe or product thereof as described herein includes an assembly for branched signal amplification. In some embodiments, the assembly complex comprises an amplifier hybridized directly or indirectly (via one or more oligonucleotides) to a sequence of the primary probe or product thereof. In some embodiments, the assembly includes one or more amplifiers each including an amplifier repeating sequence. In some aspects, the one or more amplifiers is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, detection of hybridized primary probes or products thereof includes hybridization chain reaction (HCR). HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721, all of which are herein incorporated by reference in their entireties. See also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401, all of which are herein incorporated by reference in their entireties. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, the primary probe or product thereof as described herein can be detected with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes. See, e.g., U.S. Pat. Pub. No. US20190106733, which is incorporated herein by reference in its entirety, for exemplary molecules and PER reaction components.

In some embodiments, a hybridized primary probe or product thereof as described herein can be detected by providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in an overhang region of the first and/or second probe).

In some embodiments, the methods comprise sequencing all or a portion of the hybridized primary probe or product thereof, such as one or more barcode sequences present therein. In some embodiments, the analysis and/or sequence determination comprises sequencing all or a portion of the primary probe or product thereof, and/or in situ hybridization to the primary probe or product thereof. In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference in its entirety, for exemplary probes and HCR reaction components. In some embodiments, the detection or determination comprises directly or indirectly hybridizing to the primary probe or product thereof a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some embodiments, the barcode regions or complements thereof comprised by the probes forming a hybridization complex with the target nucleic acid or products thereof are targeted directly or indirectly by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are herein incorporated by reference in their entireties. In some embodiments, these assays enable signal amplification, combinatorial decoding, and error correction schemes at the same time.

In some aspects, the provided methods comprise imaging a signal generated from the hybridized probe or product thereof, for example, via binding of the detection probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some aspects, a fluorophore comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s), probes, or products thereof (e.g. RCA products) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, each of which is herein incorporated by reference in its entirety. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some aspects, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Nucleotides having other fluorophores can be custom synthesized (See, e.g., Henegariu et al. (2000) Nature Biotechnol. 18:345, the content of which is herein incorporated by reference in its entirety).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (See, e.g., Lakowicz et al. (2003) Bio Techniques 34:62, the content of which is herein incorporated by reference in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, an antibody can be an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537, 4,849,336, and 5,073,562, the entire contents of each of which are incorporated herein by reference. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

### VII. Kits and Compositions

Provided herein is a composition for analyzing a biological sample comprising a plurality of primary probes and a library of intermediate probes (e.g., as described in Section III). In some embodiments, each primary probe comprises a binding region configured to bind to an analyte, and a barcode region associated with the analyte. In some embodiments, each intermediate probe of the library of intermediate probes comprises a hybridization region configured to hybridize to the barcode region of a primary probe of the plurality of primary probes or a complement of the barcode region, a spot-calling region, and optionally a decoding region. In some cases, two or more of the intermediate probes in the library comprise a common spot-calling region. In some cases, each intermediate probe of the library of intermediate probes comprises a decoding region. In some cases, a kit comprises a plurality of primary probes and a kit comprises a library of intermediate probes.

In some embodiments, the composition comprises detectable probes that are configured to hybridize to the spot-calling region of the intermediate probe library, and/or detectable probes that are configured to hybridize to the decoding regions of the intermediate probes. In some embodiments, the detectable probes configured to hybridize to the spot-calling region and/or the detectable probes configured to hybridize to the decoding regions are fluorescently labeled and/or configured to directly or indirectly bind to fluorescently labeled probes.

Also provided herein are kits, for analyzing an analyte in a biological sample according to any of the methods described herein (e.g., in Section III-VI). In some embodiments, the kit comprises any of the compositions or combinations thereof provided herein. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer.

### VIII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have any suitable alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Any suitable useful non-native bases that can be included in a nucleic acid or nucleotide can be used. See, for example, in Ochoa and Milam, Molecules, 25(20):4659 (2020); and McKenzie et al., Chem Soc Rev., 50(8):5126-5164 (2021), the entire contents of each of which are incorporated herein by reference.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay or an analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (Di1C18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Jr, Co, Cu, Bi, or a combination thereof.

### EXAMPLES

The following examples are included for illustrative purposes only and are not intended to limit the scope of the present disclosure.

### Example 1: Sequential Cycles for Spot-Calling and Decoding

### Primary Probe RCA and Exemplary Spot-Calling/Decoding Cycles

This example demonstrates a method of using sequential hybridization of detectably-labeled probes to detect target analytes in a sample.

A biological sample is contacted with a library of primary probes, each primary probe comprising (i) a binding region configured to bind to a target analyte, and (ii) a barcode region associated with the analyte. Each primary probe for a target analyte has a different binding region and barcode region associated with each different target analyte. The primary probes comprise circularizable probes or probe sets (e.g. padlock probes). The primary probes are hybridized to target nucleic acid analytes and ligated to generate circularized primary probes. An RCA primer is hybridized to the circularized primary probes, and an RCA reaction mixture (containing Phi29 reaction buffer, dNTPs, and Phi29 polymerase) is added to the sample. The sample is incubated at an incubation temperature (e.g., 30° C. or 37° C.) for a defined period of time (e.g. 3 hours), allowing the circularized primary probes to be amplified by the DNA polymerase to generate RCA products, each RCA product comprising multiple copies of a complement of the barcode region of the primary probe. The RCA reaction is terminated, for example by washing the sample with TE buffer.

Sequential hybridization and imaging cycles are performed to identify the locations of target nucleic acid analytes in the sample, and signals are detected and analyzed to reveal the identities of target nucleic acid analytes at the locations. The imaging comprises one or more spot-calling step(s) (generating spot-calling signals to map the locations of all or a subset of target analytes) and one or more decoding steps (generating decoding signals to determine the identities of analytes in specific locations) following each sequential hybridization of detectable probes. Sequential cycles comprising spot-calling, decoding, or both spot-calling and decoding, can be performed in any order. For example, a spot-calling step (in one or more cycles) can be performed before any decoding step is performed, between decoding steps (e.g., each cycle of the sequential hybridization of detectable probes), or after decoding steps are completed.

In an exemplary cycle comprising a spot-calling step, the sample is contacted with an intermediate probe library and detectable probes. Each intermediate probe corresponds to an analyte, and comprises (i) a hybridization region configured to hybridize to the complement of the barcode region of the primary probe corresponding to the analyte, and (ii) a spot-calling region. Fluorophore-labeled (e.g. Cy5-labeled) detectable probes are hybridized to spot-calling regions of intermediate probes. The sample is imaged to detect signals associated with the spot-calling regions (e.g., spot-calling signals) at multiple locations in the sample. The locations of the spot-calling signals are recorded. In exemplary spot-calling steps without diluted spot-calling, the intermediate probes in the intermediate probe library comprise the same spot-calling region (e.g. as shown in FIG. 1A or FIG. 1C), and a single detectable probe is used for spot-calling using a single fluorophore label (e.g. as shown in FIG. 3A). In exemplary spot-calling steps with diluted spot-calling, the intermediate probe library comprises two or more sub-libraries, each sub-library corresponds to a different subset of target analytes, and the spot-calling region of each intermediate probe is sub-library specific (e.g. as shown in FIG. 1B or FIG. 1D). Different intermediate probe sub-libraries are hybridized with different detectable probes for spot-calling, which may comprise different fluorophores (e.g. as shown in FIG. 3C). The spot-calling signals generated with diluted spot-calling (e.g., with different spot-calling regions/signals corresponding to different subsets of analytes) are less crowded than spot-calling signals generated without diluted spot-calling (e.g. as shown in FIGS. 4A-4B). Signals associated with the detected spot-calling regions and their associated locations are used for registration of signals from one or more decoding steps.

In an exemplary cycle comprising a decoding step, the sample is contacted with an intermediate probe library and detectable probes. Each intermediate probe corresponds to an analyte, and comprises (i) a hybridization region configured to hybridize to the complement of the barcode region of the primary probe corresponding to the analyte, and (ii) a decoding region. Intermediate probes comprise different decoding regions, which are hybridized by different detectable probes comprising different fluorophores. The sample is imaged, and the locations of signals associated with the decoding regions (e.g., decoding signals) are recorded. The decoding signals are assigned to locations in the sample, and may be assigned to locations of spot-calling signals detected in the current cycle or another cycle.

In an exemplary cycle comprising both a spot-calling step and a decoding step, a single intermediate probe library is used, each intermediate probe corresponds to an analyte, and each intermediate probe comprises (i) a hybridization region configured to hybridize to the complement of the barcode region of the primary probe corresponding to the analyte, (ii) a spot-calling region, and (iii) a decoding region (e.g. as shown in FIG. 1C or FIG. 1D). Detectable probes for spot-calling and decoding are hybridized to the intermediate probes and imaged. The locations of spot-calling signals and decoding signals are recorded (e.g. as shown in FIGS. 3A-3D).

### Sequential Hybridization Cycles for Decoding Target Analytes

In one illustrative example, the biological sample is contacted with 4 primary probes corresponding to 4 analytes (analytes A-D), for example as shown in FIG. 3C. The primary probes are circularizable probes (e.g. padlock probes), and comprise analyte-specific barcode regions. The primary probes are hybridized to target analytes, circularized, and used as templates for an RCA reaction, creating RCA products. The RCA products are then interrogated in sequential cycles to determine the locations and identities of the target analytes in the biological sample.

In a first cycle (e.g., cycle 1), the RCA products are contacted with and hybridized by an intermediate probe library comprising 4 intermediate probes, one for each of the 4 analytes, for example as shown in FIG. 3C. Each intermediate probe corresponds to an analyte, and each intermediate probe comprises (i) a hybridization region capable of hybridizing to the complement of the barcode region of the primary probe corresponding to the analyte, (ii) a spot-calling region, and (iii) a decoding region. The intermediate probes in cycle 1 each comprise a decoding region that can be used to generate a corresponding decoding signal designated 1, 2, or 3, as shown in Table E1. The intermediate probe library comprises a first sub-library and a second sub-library, with corresponding first and second spot-calling regions. The intermediate probe library is hybridized to fluorophore-labeled detectable probes that each comprise different fluorophores and hybridize to a corresponding spot-calling region or decoding region. The sample is imaged and the locations of spot-calling signals and decoding signals are recorded, for example as shown in FIG. 3D. Decoding signals are aligned with and/or assigned to the locations of spot-calling signals. The intermediate and detectable probes are stripped and washed from the sample.

A second and third cycle (e.g., cycle 2 and cycle 3) are performed with intermediate and detectable probes as in cycle 1, with intermediate probe libraries comprising different combinations of decoding regions for generating corresponding decoding signals as shown in Table E1, and for example as shown in FIG. 2B. In each cycle, the locations of spot-calling signals and decoding signals are recorded and decoding signals are aligned with and/or assigned to the locations of spot-calling signals.

After cycles 1-3, sequences of decoding signals recorded at the same location corresponding to a spot-calling signal are determined, and the sequence of signals recorded in a single location delineates a signal code sequence. The signal code sequence at a specific location in the biological sample corresponds to the presence of a specific analyte. For example, as shown in Table E1 and FIG. 2B, the signal code sequence of 1-2-1 indicates the presence of analyte A.

The images and signal locations from each cycle are analyzed to determine the identities, locations, and abundance of one or more of the analytes in the sample.

TABLE E1
Decoding Scheme for Analytes A-D in a Biological Sample

| | | Cycle 1 | Cycle 2 | Cycle 3 |
|---|---|---|---|---|
| | Signal Code | Decoding | Decoding | Decoding |
| Analyte | Sequence | Signal | Signal | Signal |
| Analyte A | 1-2-1 | 1 | 2 | 1 |
| Analyte B | 2-2-3 | 2 | 2 | 3 |
| Analyte C | 3-1-2 | 3 | 1 | 2 |
| Analyte D | 2-3-2 | 2 | 3 | 2 |

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a plurality of primary probes, wherein:
each primary probe comprises (i) a binding region configured to bind to an analyte, and (ii) a barcode region associated with the analyte;
(b) contacting the biological sample with a library of intermediate probes, wherein:
each intermediate probe comprises (i) a hybridization region configured to hybridize to the barcode region of a primary probe of the plurality of primary probes or a complement of the barcode region, (ii) a spot-calling region, and optionally (iii) a decoding region; and
two or more of the intermediate probes in the library comprise a common spot-calling region;
(c) detecting signals associated with the spot-calling regions of the library of intermediate probes at multiple locations in the biological sample, thereby detecting the locations of the analytes associated with the barcode regions or complements thereof hybridized by the library of intermediate probes; and
(d) detecting signals or absence thereof associated with the barcode regions or complements thereof at at least a subset of the locations detected in step (c).

2. The method of claim 1, wherein in (b), the hybridization region of the intermediate probe hybridizes to the barcode region of the primary probe.

3. The method of claim 1, comprising a step of generating a product of the primary probe after the contacting in step (a), wherein the product comprises the barcode region of the primary probe or complement thereof.

4. The method of claim 3, wherein in (b), the hybridization region of the intermediate probe hybridizes to the barcode region of the primary probe or complement thereof in the product of the primary probe.

5. The method of claim 3 or 4, wherein the product is a rolling circle amplification (RCA) product comprising multiple copies of the barcode region or complement thereof.

6. The method of any of claims 1-5, comprising one or more wash steps after the contacting in step (a), optionally wherein the one or more wash steps comprise stringent wash to remove unbound and/or nonspecifically bound primary probe molecules from the biological sample.

7. The method of any of claims 1-6, comprising one or more wash steps after the contacting in step (b), optionally wherein the one or more wash steps comprise stringent wash to remove unbound and/or nonspecifically bound intermediate probe molecules from the biological sample.

8. The method of any of claims 1-7, wherein the detecting in (c) comprises detecting signals associated with detectable probes that are hybridized to the spot-calling regions of the library of intermediate probes

9. The method of claim 8, wherein the detectable probes hybridized to the spot-calling regions are fluorescently labeled and/or configured to directly or indirectly bind to fluorescently labeled probes.

10. The method of any of claims 1-10, wherein the detecting in (d) comprises:
detecting signals associated with detectable probes that are hybridized to the barcode regions or complements thereof; and/or
detecting signals associated with detectable probes that are hybridized to the intermediate probes which are in turn hybridized to the barcode regions or complements thereof.

11. The method of claim 10, wherein the detectable probes hybridized to the barcode regions or complements thereof and/or the detectable probes hybridized to the intermediate probes which are in turn hybridized to the barcode regions or complements thereof are fluorescently labeled and/or configured to directly or indirectly bind to fluorescently labeled probes.

12. The method of any of claims 1-11, wherein the detecting in (d) comprises detecting signals associated with the decoding regions of the library of intermediate probes.

13. The method of any of claims 1-12, wherein all of the intermediate probes in the library comprise the same common spot-calling region.

14. The method of any of claims 1-12, wherein:
the library of intermediate probes comprises a first sub-library and a second sub-library,
the intermediate probes in the first sub-library comprise a first common spot-calling region,
the intermediate probes in the second sub-library comprise a second common spot-calling region, and
the first and second common spot-calling regions are different.

15. The method of claim 14, wherein the detecting in (c) comprises:
(i) detecting signals associated with the first common spot-calling region of the first sub-library of intermediate probes at multiple locations in the biological sample, thereby detecting the locations of the analytes associated with the barcode regions or complements thereof hybridized by the first sub-library of intermediate probes; and/or
(ii) detecting signals associated with the second common spot-calling region of the second sub-library of intermediate probes at multiple locations in the biological sample, thereby detecting the locations of the analytes associated with the barcode regions or complements thereof hybridized by the second sub-library of intermediate probes.

16. The method of claim 15, wherein the detecting in (c) comprises:
contacting the biological sample with detectable probes that hybridize to the first common spot-calling region in the first sub-library of intermediate probes; and/or
contacting the biological sample with detectable probes that hybridize to the second common spot-calling region in the second sub-library of intermediate probes.

17. The method of claim 15 or 16, wherein the detecting in (d) comprises:
detecting signals associated with the barcode regions or complements thereof at at least a subset of the locations detected in (c)(i); and/or
detecting signals associated with the barcode regions or complements thereof at at least a subset of the locations detected in (c)(ii).

18. The method of any of claims 14-17, wherein:
the first sub-library comprises intermediate probes configured to hybridize to the barcode regions of primary probes or complements thereof associated with two or more different analytes;
and/or the second sub-library comprises intermediate probes configured to hybridize to the barcode regions of primary probes or complements thereof associated with two or more different analytes.

19. The method of any of claims 14-18, wherein one or more intermediate probes in the first sub-library and one or more intermediate probes in the second sub-library are configured to hybridize to the barcode regions of primary probes or complements thereof associated with different analytes.

20. The method of any of claims 1-19, further comprising contacting the biological sample with a library of decoding intermediate probes, wherein each decoding intermediate probe comprises (i) a hybridization region configured to hybridize to the barcode region of a primary probe of the plurality of primary probes or a complement of the barcode region, and (ii) a decoding region.

21. The method of claim 20, comprising contacting the biological sample with detectable probes that hybridize to the decoding regions of the library of decoding intermediate probes, and detecting signals or absence thereof associated with the detectable probes hybridized to the decoding regions of the library of decoding intermediate probes.

22. The method of claim 21, wherein the detectable probes hybridized to the decoding regions of the library of decoding intermediate probes are fluorescently labeled and/or configured to directly or indirectly bind to fluorescently labeled probes.

23. The method of any of claims 20-22, further comprising contacting the biological sample with a subsequent library of decoding intermediate probes, wherein each subsequent decoding intermediate probe comprises (i) a hybridization region configured to hybridize to the barcode region of a primary probe of the plurality of primary probes or a complement of the barcode region, and (ii) a subsequent decoding region.

24. The method of claim 23, comprising contacting the biological sample with subsequent detectable probes that hybridize to the subsequent decoding regions of the subsequent library of decoding intermediate probes, and detecting signals or absence thereof associated with the subsequent detectable probes hybridized to the subsequent decoding regions.

25. The method of claim 23 or 24, wherein the decoding intermediate probes and the subsequent decoding intermediate probes hybridize to the same barcode sequence (or complement thereof) or different barcode sequences (or complements thereof) of the barcode region, optionally wherein the different barcode sequences are partially overlapping.

26. The method of any of claims 23-25, wherein the decoding region and the subsequent decoding region are configured to hybridize to the same detectable probe, optionally wherein the decoding region and the subsequent decoding region are identical in sequence and hybridize to detectable probes comprising the same detectable label.

27. The method of any of claims 23-25, wherein the decoding region and the subsequent decoding region are configured to hybridize to detectable probes of different sequences, optionally wherein each detectable probe of a different sequence comprises a different detectable label.

28. The method of any of claims 1-27, wherein the intermediate probes in the library of intermediate probes in (b) comprise decoding regions.

29. The method of claim 28, comprising contacting the biological sample with detectable probes that hybridize to the decoding regions of the library of intermediate probes and detecting signals or absence thereof associated with the detectable probes hybridized to the decoding regions of the library of intermediate probes.

30. The method of claim 29, wherein the detectable probes hybridized to the decoding regions of the library of intermediate probes are fluorescently labeled and/or configured to directly or indirectly bind to fluorescently labeled probes.

31. The method of any of claims 23-30, comprising generating a signal code sequence for one or more of the multiple locations, the signal code sequence comprising signal codes corresponding to:
the signals (or absence thereof) associated with the detectable probes hybridized to the decoding regions of the library of intermediate probes,
the signals (or absence thereof) associated with the detectable probes hybridized to the decoding regions of the library of decoding intermediate probes, and/or
the signals (or absence thereof) associated with the subsequent detectable probes hybridized to the decoding regions of the library of subsequent decoding intermediate probes;
wherein the signal code sequence corresponds to an analyte, thereby detecting the analyte at the one or more of the multiple locations.

32. The method of any of claims 1-31, wherein the barcode region comprises one or more barcode sequence or a combination of barcode sequences, and wherein the barcode sequence or the combination of barcode sequences corresponds to the analyte associated with the barcode region.

33. The method of any of claims 1-32, wherein each primary probe is independently selected from the group consisting of:
a primary probe comprising a 3′ or 5′ overhang, optionally wherein the 3′ or 5′ overhang comprises one or more barcode sequences;
a primary probe comprising a 3′ overhang and a 5′ overhang, optionally wherein the 3′ overhang and the 5′ overhang each independently comprises one or more barcode sequences;
a circular primary probe; and
a circularizable primary probe or probe set.

34. The method of any of claims 1-33, wherein each intermediate probe is independently selected from the group consisting of:
an intermediate primary probe comprising a 3′ or 5′ overhang, optionally wherein the 3′ or 5′ overhang comprises one or more spot-calling regions and/or decoding regions;
an intermediate probe comprising a 3′ overhang and a 5′ overhang, optionally wherein the 3′ overhang and the 5′ overhang each independently comprise one or more spot-calling regions and/or decoding regions;
a circular intermediate probe; and
a circularizable intermediate probe or probe set.

35. The method of any of claims 1-34, wherein the analyte comprises a nucleic acid, a protein, a carbohydrate, a lipid, or a small molecule, or a complex of any of the foregoing.

36. The method of any of claims 1-35, wherein the analyte is a genomic DNA, cDNA, or mRNA, and the primary probe hybridizes to the genomic DNA, cDNA, or mRNA.

37. The method of any of claims 1-35, wherein the analyte is a protein, and the primary probe comprises a binding region and one or more reporter oligonucleotides conjugated thereto, wherein the binding region binds the protein and the one or more reporter oligonucleotides comprise the barcode region.

38. The method of any of claims 1-37, wherein the biological sample is non-homogenized and optionally selected from the group consisting of a formalin-fixed, paraffin-embedded (FFPE) sample, a frozen tissue sample, and a fresh tissue sample.

39. The method of any of claims 1-38, wherein the biological sample is fixed or not fixed.

40. The method of any of claims 1-39, wherein the biological sample is permeabilized.

41. The method of any of claims 1-40, wherein the biological sample is embedded in a matrix, optionally wherein the matrix comprises a hydrogel.

42. The method of any of claims 1-41, wherein the biological sample is cleared, optionally wherein the clearing comprises contacting the biological sample with a proteinase.

43. The method of any of claims 1-42, wherein the biological sample is crosslinked.

44. The method of any of claims 1-43, wherein the biological sample is a tissue slice between about 1 μm and about 50 μm in thickness, optionally wherein the tissue slice is between about 5 μm and about 35 μm in thickness.

45. A method for analyzing a biological sample, comprising:
(a) contacting the biological sample with a plurality of primary probes, wherein:
each primary probe comprises (i) a binding region configured to bind to an analyte, and (ii) a barcode region associated with the analyte; and
each primary probe is circular or circularizable;
(b) generating rolling circle amplification (RCA) products of the primary probes, wherein each RCA product comprises a complement of the barcode region of the primary probe;
(c) contacting the biological sample with a library of intermediate probes, wherein:
each intermediate probe comprises (i) a hybridization region configured to hybridize to the complement of the barcode region of a primary probe, (ii) a spot-calling region, and (iii) a decoding region; and
two or more of the intermediate probes in the library comprise a common spot-calling region;
(d) detecting signals associated with the spot-calling regions of the library of intermediate probes at multiple locations in the biological sample, thereby detecting the locations of the analytes associated with the complements of the barcode regions hybridized by the library of intermediate probes; and
(e) detecting signals or absence thereof associated with the decoding regions of the library of intermediate probes at at least a subset of the locations detected in step (d).

46. The method of claim 45, wherein all of the intermediate probes in the library comprise the same common spot-calling region.

47. The method of claim 45, wherein:
the library of intermediate probes comprises a first sub-library and a second sub-library;
the intermediate probes in the first sub-library comprise a first common spot-calling region;
the intermediate probes in the second sub-library comprise a second common spot-calling region; and
the first and second common spot-calling regions are different.

48. The method of any of claims 45-47, wherein:
a unique signal code sequence is assigned to each analyte to be analyzed;
each signal code sequence is derivable by interrogating the complements of the barcode regions with sequential intermediate probe libraries;
the method further comprises one or more sequential cycles, each cycle comprising:
(i) contacting the biological sample with a subsequent intermediate probe library, wherein each intermediate probe in the subsequent intermediate probe library comprises (a) a hybridization region configured to hybridize to the complement of the barcode region of a primary probe, (b) a decoding region, and optionally (c) a spot-calling region;
(ii) optionally detecting signals associated with the spot-calling region of the subsequent intermediate probe library at multiple locations in the biological sample, thereby detecting the locations of the analytes associated with the complements of the barcode regions hybridized by the subsequent intermediate probe library; and
(iii) detecting signals or absence thereof associated with the decoding regions of intermediate probes in the subsequent intermediate probe library at at least a subset of the locations detected in step (c) and/or step (ii);
a sequence of signals sequentially detected at at least a subset of the multiple locations in the biological sample in the sequential cycles delineates a signal code sequence corresponding to a particular analyte, thereby revealing the location of the analyte in the biological sample; and
the locations of one or more of the analytes in the biological sample are determined.

49. A method for analyzing a biological sample, comprising:
(a) contacting the biological sample with a plurality of primary probes, wherein:
each primary probe comprises (i) a binding region configured to bind to an analyte, and (ii) a barcode region associated with the analyte; and
each primary probe is circular or circularizable;
(b) generating rolling circle amplification (RCA) products of the primary probes, wherein each RCA product comprises a complement of the barcode region of the primary probe;
(c) contacting the biological sample with a first library of intermediate probes for a first plurality of analytes, wherein:
each intermediate probe of the first library comprises (i) a hybridization region configured to hybridize to the complement of the barcode region of a primary probe, (ii) a first common spot-calling region, and (iii) a decoding region;
(d) detecting signals associated with the first common spot-calling regions at first multiple locations in the biological sample, thereby detecting the locations of the analytes associated with the complements of the barcode regions hybridized by the first library of intermediate probes;
(e) detecting signals or absence thereof associated with the decoding regions of the first library of intermediate probes at at least a subset of the first multiple locations detected in step (d), in order to detect the first plurality of analytes;
(f) contacting the biological sample with a second library of intermediate probes for a second plurality of analytes, wherein:
each intermediate probe of the second library comprises (i) a hybridization region configured to hybridize to the complement of the barcode region of a primary probe, (ii) a second common spot-calling region, and (iii) a decoding region;
(g) detecting signals associated with the second common spot-calling regions at second multiple locations in the biological sample, thereby detecting the locations of the analytes associated with the complements of the barcode regions hybridized by the second library of intermediate probes; and
(h) detecting signals or absence thereof associated with the decoding regions of the second library of intermediate probes at at least a subset of the second multiple locations detected in step (g), in order to detect the second plurality of analytes.

50. The method of claim 49, wherein the first plurality of analytes and the second plurality of analytes do not comprise a common analyte or comprise one or more common analytes.

51. The method of claim 49 or 50, wherein the first multiple locations and the second multiple locations do not spatially overlap or comprise no more than 1%, no more than 5%, no more than 10%, no more than 20%, or no more than 30% spatially overlapping locations.

52. The method of any of claims 1-51, comprising aligning the signals associated with the spot-calling regions with the signals or absence thereof associated with the barcode regions or complements thereof.

53. The method of any of claims 1-51, wherein the signals associated with the spot-calling regions are used for registration of the signals or absence thereof associated with the barcode regions or complements thereof.

54. The method of any of claims 12-51, comprising aligning the signals associated with the spot-calling regions with the signals or absence thereof associated with the decoding regions.

55. The method of any of claims 12-51, wherein the signals associated with the spot-calling regions are used for registration of the signals or absence thereof associated with the decoding regions.

56. The method of claim 53 or 55, wherein the registration comprises associating the signals or absence thereof associated with the decoding region or barcode regions or complements thereof with the locations of detected signals associated with the spot-calling regions.

57. A kit for analyzing a biological sample, comprising:
(a) a plurality of primary probes, wherein:
each primary probe comprises (i) a binding region configured to bind to an analyte, and (ii) a barcode region associated with the analyte; and
(b) a library of intermediate probes, wherein:
each intermediate probe comprises (i) a hybridization region configured to hybridize to the barcode region of a primary probe of the plurality of primary probes or a complement of the barcode region, (ii) a spot-calling region, and optionally (iii) a decoding region; and
two or more of the intermediate probes in the library comprise a common spot-calling region.

58. The kit of claim 57, wherein each intermediate probe comprises a decoding region.

59. The kit of claim 57 or 58, further comprising detectable probes that are configured to hybridize to the spot-calling region of the intermediate probe library, and/or detectable probes that are configured to hybridize to the decoding regions of the intermediate probes.

60. The kit of claim 59, wherein the detectable probes configured to hybridize to the spot-calling region and/or the detectable probes configured to hybridize to the decoding regions are fluorescently labeled and/or configured to directly or indirectly bind to fluorescently labeled probes.
